SEQUENCE LISTING (61 sequences)

SEQ ID NO: 1 - DNA, 33 bp, Mycobacterium tuberculosis
acccaactgaatggagccaccgccgtgtccgac

SEQ ID NO: 2 - DNA, 38 bp, Mycobacterium tuberculosis
acgcacttgacttgtcttcaggaccaccacccgaggat

SEQ ID NO: 3 - DNA, 42 bp, Mycobacterium tuberculosis
acccaactgaatggagccgttcatctcgttggctaccgtgtt

SEQ ID NO: 4 - DNA, 37 bp, Mycobacterium tuberculosis
acgcacttgacttgtcttcccttgacgccgacgctgt

SEQ ID NO: 5 - DNA, 35 bp, Mycobacterium tuberculosis
acccaactgaatggagcacagcgtcggcgtcaagg

SEQ ID NO: 6 - DNA, 42 bp, Mycobacterium tuberculosis
acgcacttgacttgtcttctgtggacgaccagcagaacactg

SEQ ID NO: 7 - DNA, 35 bp, Mycobacterium tuberculosis
acccaactgaatggagcgagccagaacgccgagtc

SEQ ID NO: 8 - DNA, 41 bp, Mycobacterium tuberculosis
acgcacttgacttgtcttcggtcccatagcctaccgacttc

SEQ ID NO: 9 - DNA, 39 bp, Mycobacterium tuberculosis
acccaactgaatggagcgaagtcggtaggctatgggacc

SEQ ID NO: 10 - DNA, 37 bp, Mycobacterium tuberculosis
acgcacttgacttgtcttcccccgcacgatctcaacg

SEQ ID NO: 11 - DNA, 41 bp, Mycobacterium tuberculosis
acccaactgaatggagcggggcatggccgacaaacagaacg

SEQ ID NO: 12 - DNA, 43 bp, Mycobacterium tuberculosis
acgcacttgacttgtcttcctcgccgggaatgtacgccgtgac

SEQ ID NO: 13 - DNA, 45 bp, Mycobacterium tuberculosis
acccaactgaatggagcaaggttgcccgcgtgaagttgacgagtc

SEQ ID NO: 14 - DNA, 41 bp, Mycobacterium tuberculosis
acgcacttgacttgtcttcgggccccttgcgtggcatcagc

SEQ ID NO: 15 - DNA, 44 bp, Mycobacterium tuberculosis
acccaactgaatggagccgcaagtccgaactgtatgtcgtagaa

SEQ ID NO: 16 - DNA, 39 bp, Mycobacterium tuberculosis
acccaactgaatggagcgagagttggtgcggcgtaagag

SEQ ID NO: 17 - DNA, 43 bp, Mycobacterium tuberculosis
acgcacttgacttgtcttcccatcagcacgatctkgtggtagc

SEQ ID NO: 18 - DNA, 35 bp, Mycobacterium tuberculosis
acccaactgaatggagcacgctccggtgtaggcac

SEQ ID NO: 19 - DNA, 37 bp, Mycobacterium tuberculosis
acgcacttgacttgtcttctatagggtccatgacgcc

SEQ ID NO: 20 - DNA, 35 bp, Mycobacterium tuberculosis
acccaactgaatggagcgtgaacaacccgacccag

SEQ ID NO: 21 - DNA, 38 bp, Mycobacterium tuberculosis
acgcacttgacttgtcttcgcctcgattgccgacgtgt

SEQ ID NO: 22 - DNA, 41 bp, Mycobacterium tuberculosis
acccaactgaatggagcctgccgcgtcggtaggcaaactgc

SEQ ID NO: 23 - DNA, 42 bp, Mycobacterium tuberculosis
acccaactgaatggagcctggccgaagcggcggactaccatc

SEQ ID NO: 24 - DNA, 42 bp, Mycobacterium tuberculosis
acccaactgaatggagcgtactcccggcgcggacttccatcc

SEQ ID NO: 25 - DNA, 40 bp, Mycobacterium tuberculosis
acccaactgaatggagcggcaacgcggcgtcgatgaggtc

SEQ ID NO: 26 - DNA, 41 bp, Mycobacterium tuberculosis
acgcacttgacttgtcttcccgcgccgggagkaccgctgac

SEQ ID NO: 27 - DNA, 45 bp, Mycobacterium tuberculosis
acgcacttgacttgtcttcggcgtgccgttctcgtcgactccttc

SEQ ID NO: 28 - DNA, 44 bp, Mycobacterium tuberculosis
acgcacttgacttgtcttccctggtggccaagccattgcgtacc

SEQ ID NO: 29 - DNA, 40 bp, Mycobacterium tuberculosis
acgcacttgacttgtcttcgcgctccaccgccgccaacag

SEQ ID NO: 30 - DNA, 35 bp, Mycobacterium tuberculosis
acccaactgaatggagcgctgattccggcaagctg

SEQ ID NO: 31 - DNA, 39 bp, Mycobacterium tuberculosis
acgcacttgacttgtcttcgtggatgcgcctgccagacc

SEQ ID NO: 32 - DNA, 38 bp, Mycobacterium tuberculosis
acccaactgaatggagccccatgtcagcgacgccagtc

SEQ ID NO: 33 - DNA, 43 bp, Mycobacterium tuberculosis
acgcacttgacttgtcttccgctggtcacctatgtgctgatcg

SEQ ID NO: 34 - DNA, 41 bp, Mycobacterium tuberculosis
acccaactgaatggagcggccacggccggctagcacctctt

SEQ ID NO: 35 - DNA, 44 bp, Mycobacterium tuberculosis
acgcacttgacttgtcttcgagcttgctgaacgccgcgatctcg

SEQ ID NO: 36 - DNA, 41 bp, Mycobacterium tuberculosis; identical to SEQ ID NO: 34

SEQ ID NO: 37 - DNA, 43 bp, Mycobacterium tuberculosis
acgcacttgacttgtcttcgggctgcttggcgtcgaccttgga

SEQ ID NO: 38 - DNA, 47 bp, Mycobacterium tuberculosis
acccaactgaatggagcgcaaccaaatgcattgtccgctttgatgat

SEQ ID NO: 39 - DNA, 44 bp, Mycobacterium tuberculosis
acgcacttgacttgtcttctgctgaacgccgcgatctcggtagg

SEQ ID NO: 40 - DNA, 45 bp, Mycobacterium tuberculosis
acccaactgaatggagcggccagcgtcgacgtcaacatccagtag

SEQ ID NO: 41 - DNA, 40 bp, Mycobacterium tuberculosis
acccaactgaatggagcgcacgcccgaacgcgacggttat

SEQ ID NO: 42 - DNA, 40 bp, Mycobacterium tuberculosis
acccaactgaatggagcgcggagctgcggctggacgactc

SEQ ID NO: 43 - DNA, 36 bp, Mycobacterium tuberculosis
acccaactgaatggagcacgccggcgcgggtgttca

SEQ ID NO: 44 - DNA, 43 bp, Mycobacterium tuberculosis
acgcacttgacttgtcttcccagccgcagctccgccaggtatc

SEQ ID NO: 45 - DNA, 45 bp, Mycobacterium tuberculosis
acgcacttgacttgtcttccggcgaagatctccgcggtcaactct

SEQ ID NO: 46 - DNA, 41 bp, Mycobacterium tuberculosis
acgcacttgacttgtcttccggccatccgggtgcccttgaa

SEQ ID NO: 47 - DNA, 46 bp, Mycobacterium tuberculosis
acgcacttgacttgtcttcgccggcgtcttgacgtcgattttgagt

SEQ ID NO: 48 - DNA, 561 bp, Mycobacterium tuberculosis
atgcgggcgttgatcatcgtcgacgtgcagaacgacttctgcgagggtggctcgctggcggtaaccggtggcgccgcgctggcccgcgccatcagcgactacctggccgaagcggcggactaccatcacgtcgtggcaaccaaggacttccacatcgacccgggtgaccacttctccggcacaccggactattcctcgtcgtggccaccgcattgcgtcagcggtactcccggcgcggacttccatcccagtctggacacgtcggcaatcgaggcggtgttctacaagggtgcctacaccggagcgtacagcggcttcgaaggagtcgacgagaacggcacgccactgctgaattggctgcggcaacgcggcgtcgatgaggtcgatgtggtcggtattgccaccgatcattgtgtgcgccagacggccgaggacgcggtacgcaatggcttggccaccagggtgctggtggacctgacagcgggtgtgtcggccgataccaccgtcgccgcgctggaggagatgcgcaccgccagcgtcgagttggtttgcagctcctga

SEQ ID NO: 49 - DNA, 807 bp, Mycobacterium tuberculosis
gtggcacgacgtgcccgcgttgacgccgagctagtccggcggggcctggcgcgatcacgtcaacaggccgcggagttgatcggcgccggcaaggtgcgcatcgacgggctgccggcggtcaagccggccaccgccgtgtccgacaccaccgcgctgaccgtggtgaccgacagtgaacgcgcctgggtatcgcgcggagcgcacaaactagtcggtgcgctggaggcgttcgcgatcgcggtggcgggccggcgctgtctggacgcgggcgcatcgaccggtgggttcaccgaagtactgctggaccgtggtgccgcccacgtggtggccgccgatgtcggatacggccagctggcgtggtcgctgcgcaacgatcctcgggtggtggtcctcgagcggaccaacgcacgtggcctcacaccggaggcgatcggcggtcgcgtcgacctggtagtggccgacctgtcgttcatctcgttggctaccgtgttgcccgcgctggttggatgcgcttcgcgcgacgccgatatcgttccactggtgaagccgcagtttgaggtggggaaaggtcaggtcggccccggtggggtggtccatgacccgcagttgcgtgcgcggtcggtgctcgcggtcgcgcggcgggcacaggagctgggctggcacagcgtcggcgtcaaggccagcccgctgccgggcccatcgggcaatgtcgagtacttcctgtggttgcgcacgcagaccgaccgggcattgtcggccaagggattggaggatgcggtgcaccgtgcgattagcgagggcccgtag

SEQ ID NO: 50 - DNA, 675 bp, Mycobacterium tuberculosis
tcacgccgtccctccactcgccatccgtgccgaccctcgggcgatctgctttccacgtcgtgcgaacaccacggtcgcgggcggacgcaaatagttcgcgccacatgtcaccaccctgacatcaaccgcgcccgatgcgatcatcacacgccggtgctcccgtacttcgtcgtgagcccgctcgcctttgatggcgagcattcgcccgttcggccgtatcaacggcatgctccatttcgtcaacttgtccaacgcggccaccgcccgtgacaccgcagcgtcgctgccgcccaattggtcctgcacccaggactcctcggcgcgcccccgcacgatctcaacggccacgcccagatctgtcaccatctctcgaagaaactcggtgcggcgcagtagcggttctaggagaactacctggaggtccggccgcgctatcgccaatggcacgcccggcaacccggctccgctaccgatatccacgacccggtcaccgcgttcgaggagctcaccgatcacggcgcagttcagtagatgccggtcccatagcctaccgacttcgcggggtcccaccagcccccgctccacaccgggtcccgccaacgcttcggcgtaccgccgagcaaggccaagccgcggtccgaagatcgcagacgccgcgggctcgatcggagacat

SEQ ID NO: 51 - DNA, 375 bp, Mycobacterium tuberculosis
atgccaaccatccagcagctggtccgcaagggtcgtcgggacaagatcagtaaggtcaagaccgcggctctgaagggcagcccgcagcgtcgtggtgtatgcacccgcgtgtacaccaccactccgaagaagccgaactcggcgcttcggaaggttgcccgcgtgaagttgacgagtcaggtcgaggtcacggcgtacattcccggcgagggccacaacctgcaggagcactcgatggtgctggtgcgcggcggccgggtgaaggacctgcctggtgtgcgctacaagatcatccgcggttcgctggatacgcagggtgtcaagaaccgcaaacaggcacgcagccgttacggcgctaagaaggagaagggctga

SEQ ID NO: 52 - DNA, 2028 bp, Mycobacterium tuberculosis
gtggctgcccagaaaaagaaggcccaagacgaatacggcgctgcgtctatcaccattctcgaagggctggaggccgtccgcaaacgtcccggcatgtacattggctcgaccggtgagcgcggtttacaccatctcatttgggaggtggtcgacaacgcggtcgacgaggcgatggccggttatgcaaccacagtgaacgtagtgctgcttgaggatggcggtgtcgaggtcgccgacgacggccgcggcattccggtcgccacccacgcctccggcataccgaccgtcgacgtggtgatgacacaactacatgccggcggcaagttcgactcggacgcgtatgcgatatctggtggtctgcacggcgtcggcgtgtcggtggttaacgcgctatccacccggctcgaagtcgagatcaagcgcgacgggtacgagtggtctcaggtttatgagaagtcggaacccctgggcctcaagcaaggggcgccgaccaagaagacggggtcaacggtgcggttctgggccgaccccgctgttttcgaaaccacggaatacgacttcgaaaccgtcgcccgccggctgcaagagatggcgttcctcaacaaggggctgaccatcaacctgaccgacgagagggtgacccaagacgaggtcgtcgacgaagtggtcagcgacgtcgccgaggcgccgaagtcggcaagtgaacgcgcagccgaatccactgcaccgcacaaagttaagagccgcacctttcactatccgggtggcctggtggacttcgtgaaacacatcaaccgcaccaagaacgcgattcatagcagcatcgtggacttttccggcaagggcaccgggcacgaggtggagatcgcgatgcaatggaacgccgggtattcggagtcggtgcacaccttcgccaacaccatcaacacccacgagggcggcacccacgaagagggcttccgcagcgcgctgacgtcggtggtgaacaagtacgccaaggaccgcaagctactgaaggacaaggaccccaacctcaccggtgacgatatccgggaaggcctggccgctgtgatctcggtgaaggtcagcgaaccgcagttcgagggccagaccaagaccaagttgggcaacaccgaggtcaaatcgtttgtgcagaaggtctgtaacgaacagctgacccactggtttgaagccaaccccaccgacgcgaaagtcgttgtgaacaaggctgtgtcctcggcgcaagcccgtatcgcggcacgtaaggcacgagagttggtgcggcgtaagagcgccaccgacatcggtggattgcccggcaagctggccgattgccgttccacggatccgcgcaagtccgaactgtatgtcgtagaaggtgactcggccggcggttctgcaaaaagcggtcgcgattcgatgttccaggcgatacttccgctgcgcggcaagatcatcaatgtggagaaagcgcgcatcgaccgggtgctaaagaacaccgaagttcaggcgatcatcacggcgctgggcaccgggatccacgacgagttcgatatcggcaagctgcgctaccacaagatcgtgctgatggccgacgccgatgttgacggccaacatatttccacgctgttgttgacgttgttgttccggttcatgcggccgctcatcgagaacgggcatgtgtttttggcacaaccgccgctgtacaaactcaagtggcagcgcagtgacccggaattcgcatactccgaccgcgagcgcgacggtctgctggaggcggggctgaaggccgggaagaagatcaacaaggaagacggcattcagcggtacaagggtctaggtgaaatggacgctaaggagttgtgggagaccaccatggatccctcggttcgtgtgttgcgtcaagtgacgctggacgacgccgccgccgccgacgagttgttctccatcctgatgggcgaggacgtcgacgcgcggcgcagctttatcacccgcaacgccaaggatgttcggttcctggatgtctaa

SEQ ID NO: 53 - DNA, 3297 bp, Mycobacterium tuberculosis
atgacacagtgcgcgagcagacgcaaaagcaccccaaatcgggcgattttgggggcttttgcgtctgctcgcgggacgcgctgggtggccaccatcgccgggctgattggctttgtgttgtcggtggcgacgccgctgctgcccgtcgtgcagaccaccgcgatgctcgactggccacagcgggggcaactgggcagcgtgaccgccccgctgatctcgctgacgccggtcgactttaccgccaccgtgccgtgcgacgtggtgcgcgccatgccacccgcgggcggggtggtgctgggcaccgcacccaagcaaggcaaggacgccaatttgcaggcgttgttcgtcgtcgtcagcgcccagcgcgtggacgtcaccgaccgcaacgtggtgatcttgtccgtgccgcgcgagcaggtgacgtccccgcagtgtcaacgcatcgaggtcacctctacccacgccggcaccttcgccaacttcgtcgggctcaaggacccgtcgggcgcgccgctgcgcagcggcttccccgaccccaacctgcgcccgcagattgtcggggtgttcaccgacctgaccgggcccgcgccgcccgggctggcggtctcggcgaccatcgacacccggttctccacccggccgaccacgctgaaactgctggcgatcatcggggcgatcgtggccaccgtcgtcgcactgatcgcgttgtggcgcctggaccagttggacgggcggggctcaattgcccagctcctcctcaggccgttccggcctgcatcgtcgccgggcggcatgcgccggctgattccggcaagctggcgcaccttcaccctgaccgacgccgtggtgatattcggcttcctgctctggcatgtcatcggcgcgaattcgtcggacgacggctacatcctgggcatggcccgagtcgccgaccacgccggctacatgtccaactatttccgctggttcggcagcccggaggatcccttcggctggtattacaacctgctggcgctgatgacccatgtcagcgacgccagtctgtggatgcgcctgccagacctggccgccgggctagtgtgctggctgctgctgtcgcgtgaggtgctgccccgcctcgggccggcggtggaggccagcaaacccgcctactgggcggcggccatggtcttgctgaccgcgtggatgccgttcaacaacggcctgcggccggagggcatcatcgcgctcggctcgctggtcacctatgtgctgatcgagcggtccatgcggtacagccggctcacaccggcggcgctggccgtcgttaccgccgcattcacactgggtgtgcagcccaccggcctgatcgcggtggccgcgctggtggccggcggccgcccgatgctgcggatcttggtgcgccgtcatcgcctggtcggcacgttgccgttggtgtcgccgatgctggccgccggcaccgtcatcctgaccgtggtgttcgccgaccagaccctgtcaacggtgttggaagccaccagggttcgcgccaaaatcgggccgagccaggcgtggtataccgagaacctgcgttactactacctcatcctgcccaccgtcgacggttcgctgtcgcggcgcttcggctttttgatcaccgcgctatgcctgttcaccgcggtgttcatcatgttgcggcgcaagcgaattcccagcgtggcccgcggaccggcgtggcggctgatgggcgtcatcttcggcaccatgttcttcctgatgttcacgcccaccaagtgggtgcaccacttcgggctgttcgccgccgtaggggcggcgatggccgcgctgacgacggtgttggtatccccatcggtgctgcgctggtcgcgcaaccggatggcgttcctggcggcgttattcttcctgctggcgttgtgttgggccaccaccaacggctggtggtatgtctccagctacggtgtgccgttcaacagcgcgatgccgaagatcgacgggatcacagtcagcacaatctttttcgccctgtttgcgatcgccgccggctatgcggcctggctgcacttcgcgccccgcggcgccggcgaagggcggctgatccgcgcgctgacgacagccccggtaccgatcgtggccggtttcatggcggcggtgttcgtcgcgtccatggtggccgggatcgtgcgacagtacccgacctactccaacggctggtccaacgtgcgggcgtttgtcggcggctgcggactggccgacgacgtactcgtcgagcctgataccaatgcgggtttcatgaagccgctggacggcgattcgggttcttggggccccttgggcccgctgggtggagtcaacccggtcggcttcacgcccaacggcgtaccggaacacacggtggccgaggcgatcgtgatgaaacccaaccagcccggcaccgactacgactgggatgcgccgaccaagctgacgagtcctggcatcaatggttctacggtgccgctgccctatgggctcgatcccgcccgggtaccgttggcaggcacctacaccaccggcgcacagcaacagagcacactcgtctcggcgtggtatctcctgcctaagccggacgacgggcatccgctggtcgtggtgaccgccgcgggcaagatcgccggcaacagcgtgctgcacgggtacacccccgggcagactgtggtgctcgaatacgccatgccgggacccggagcgctggtacccgccgggcggatggtgcccgacgacctatacggagagcagcccaaggcgtggcgcaacctgcgcttcgcccgagcaaagatgcccgccgatgccgtcgcggtccgggtggtggccgaggatctgtcgctgacaccggaggactggatcgcggtgaccccgccgcgggtaccggacctgcgctcactgcaggaatatgtgggctcgacgcagccggtgctgctggactgggcggtcggtttggccttcccgtgccagcagccgatgctgcacgccaatggcatcgccgaaatcccgaagttccgcatcacaccggactactcggctaagaagctggacaccgacacgtgggaagacggcactaacggcggcctgctcgggatcaccgacctgttgctgcgggcccacgtcatggccacctacctgtcccgcgactgggcccgcgattggggttccctgcgcaagttcgacaccctggtcgatgcccctcccgcccagctcgagttgggcaccgcgacccgcagcggcctgtggtcaccgggcaagatccgaattggtccatag

SEQ ID NO: 54 - DNA, 654 bp, Mycobacterium tuberculosis
atggcacgaaagggcattctcggtaccaagctgggtatgacgcaggtattcgacgaaagcaacagagtagtaccggtgaccgtggtcaaggccgggcccaacgtggtaacccgcatccgcacgcccgaacgcgacggttatagcgccgtgcagctggcctatggcgagatcagcccacgcaaggtcaacaagccgctgacaggtcagtacaccgccgccggcgtcaacccacgccgatacctggcggagctgcggctggacgactcggatgccgcgaccgagtaccaggttgggcaagagttgaccgcggagatcttcgccgatggcagctacgtcgatgtgacgggtacctccaagggcaaaggtttcgccggcaccatgaagcggcacggcttccgcggtcagggcgccagtcacggtgcccaggcggtgcaccgccgtccgggctccatcggcggatgtgccacgccggcgcgggtgttcaagggcacccggatggccgggcggatgggcaatgaccgggtgaccgttcttaaccttttggtgcataaggtcgatgccgagaacggcgtgctgctgatcaagggtgcggttcctggccgcaccggtggactggtcatggtccgcagtgcgatcaaacgaggtgagaagtga

SEQ ID NO: 55 - protein, 186 aa, Mycobacterium tuberculosis
MRALIIVDVQNDFCEGGSLAVTGGAALARAISDYLAEAADYHHVVATKDFHIDPGDHFSGTPDYSSSWPPHCVSGTPGADFHPSLDTSAIEAVFYKGAYTGAYSGFEGVDENGTPLLNWLRQRGVDEVDVVGIATDHCVRQTAEDAVRNGLATRVLVDLTAGVSADTTVAALEEMRTASVELVCSS

SEQ ID NO: 56 - protein, 268 aa, Mycobacterium tuberculosis
MARRARVDAELVRRGLARSRQQAAELIGAGKVRIDGLPAVKPATAVSDTTALTVVTDSERAWVSRGAHKLVGALEAFAIAVAGRRCLDAGASTGGFTEVLLDRGAAHVVAADVGYGQLAWSLRNDPRVVVLERTNARGLTPEAIGGRVDLVVADLSFISLATVLPALVGCASRDADIVPLVKPQFEVGKGQVGPGGVVHDPQLRARSVLAVARRAQELGWHSVGVKASPLPGPSGNVEYFLWLRTQTDRALSAKGLEDAVHRAISEGP

SEQ ID NO: 57 - protein, 224 aa, Mycobacterium tuberculosis
MSPIEPAASAIFGPRLGLARRYAEALAGPGVERGLVGPREVGRLWDRHLLNCAVIGELLERGDRVVDIGSGAGLPGVPLAIARPDLQVVLLEPLLRRTEFLREMVTDLGVAVEIVRGRAEESWVQDQLGGSDAAVSRAVAALDKLTKWSMPLIRPNGRMLAIKGERAHDEVREHRRVMIASGAVDVRVVTCGANYLRPPATVVFARRGKQIARGSARMASGGTA

SEQ ID NO: 58 - protein, 124 aa, Mycobacterium tuberculosis
MPTIQQLVRKGRRDKISKVKTAALKGSPQRRGVCTRVYTTTPKKPNSALRKVARVKLTSQVEVTAYIPGEGHNLQEHSMVLVRGGRVKDLPGVRYKIIRGSLDTQGVKNRKQARSRYGAKKEKG

SEQ ID NO: 59 - protein, 675 aa, Mycobacterium tuberculosis
MAAQKKKAQDEYGAASITILEGLEAVRKRPGMYIGSTGERGLHHLIWEVVDNAVDEAMAGYATTVNVVLLEDGGVEVADDGRGIPVATHASGIPTVDVVMTQLHAGGKFDSDAYAISGGLHGVGVSVVNALSTRLEVEIKRDGYEWSQVYEKSEPLGLKQGAPTKKTGSTVRFWADPAVFETTEYDFETVARRLQEMAFLNKGLTINLTDERVTQDEVVDEVVSDVAEAPKSASERAAESTAPHKVKSRTFHYPGGLVDFVKHINRTKNAIHSSIVDFSGKGTGHEVEIAMQWNAGYSESVHTFANTINTHEGGTHEEGFRSALTSVVNKYAKDRKLLKDKDPNLTGDDIREGLAAVISVKVSEPQFEGQTKTKLGNTEVKSFVQKVCNEQLTHWFEANPTDAKVVVNKAVSSAQARIAARKARELVRRKSATDIGGLPGKLADCRSTDPRKSELYVVEGDSAGGSAKSGRDSMFQAILPLRGKIINVEKARIDRVLKNTEVQAIITALGTGIHDEFDIGKLRYHKIVLMADADVDGQHISTLLLTLLFRFMRPLIENGHVFLAQPPLYKLKWQRSDPEFAYSDRERDGLLEAGLKAGKKINKEDGIQRYKGLGEMDAKELWETTMDPSVRVLRQVTLDDAAAADELFSILMGEDVDARRSFITRNAKDVRFLDV

SEQ ID NO: 60 - protein, 1098 aa, Mycobacterium tuberculosis
MTQCASRRKSTPNRAILGAFASARGTRWVATIAGLIGFVLSVATPLLPVVQTTAMLDWPQRGQLGSVTAPLISLTPVDFTATVPCDVVRAMPPAGGVVLGTAPKQGKDANLQALFVVVSAQRVDVTDRNVVILSVPREQVTSPQCQRIEVTSTHAGTFANFVGLKDPSGAPLRSGFPDPNLRPQIVGVFTDLTGPAPPGLAVSATIDTRFSTRPTTLKLLAIIGAIVATVVALIALWRLDQLDGRGSIAQLLLRPFRPASSPGGMRRLIPASWRTFTLTDAVVIFGFLLWHVIGANSSDDGYILGMARVADHAGYMSNYFRWFGSPEDPFGWYYNLLALMTHVSDASLWMRLPDLAAGLVCWLLLSREVLPRLGPAVEASKPAYWAAAMVLLTAWMPFNNGLRPEGIIALGSLVTYVLIERSMRYSRLTPAALAVVTAAFTLGVQPTGLIAVAALVAGGRPMLRILVRRHRLVGTLPLVSPMLAAGTVILTVVFADQTLSTVLEATRVRAKIGPSQAWYTENLRYYYLILPTVDGSLSRRFGFLITALCLFTAVFIMLRRKRIPSVARGPAWRLMGVIFGTMFFLMFTPTKWVHHFGLFAAVGAAMAALTTVLVSPSVLRWSRNRMAFLAALFFLLALCWATTNGWWYVSSYGVPFNSAMPKIDGITVSTIFFALFAIAAGYAAWLHFAPRGAGEGRLIRALTTAPVPIVAGFMAAVFVASMVAGIVRQYPTYSNGWSNVRAFVGGCGLADDVLVEPDTNAGFMKPLDGDSGSWGPLGPLGGVNPVGFTPNGVPEHTVAEAIVMKPNQPGTDYDWDAPTKLTSPGINGSTVPLPYGLDPARVPLAGTYTTGAQQQSTLVSAWYLLPKPDDGHPLVVVTAAGKIAGNSVLHGYTPGQTVVLEYAMPGPGALVPAGRMVPDDLYGEQPKAWRNLRFARAKMPADAVAVRVVAEDLSLTPEDWIAVTPPRVPDLRSLQEYVGSTQPVLLDWAVGLAFPCQQPMLHANGIAEIPKFRITPDYSAKKLDTDTWEDGTNGGLLGITDLLLRAHVMATYLSRDWARDWGSLRKFDTLVDAPPAQLELGTATRSGLWSPGKIRIGP

SEQ ID NO: 61 - protein, 217 aa, Mycobacterium tuberculosis
MARKGILGTKLGMTQVFDESNRVVPVTVVKAGPNVVTRIRTPERDGYSAVQLAYGEISPRKVNKPLTGQYTAAGVNPRRYLAELRLDDSDAATEYQVGQELTAEIFADGSYVDVTGTSKGKGFAGTMKRHGFRGQGASHGAQAVHRRPGSIGGCATPARVFKGTRMAGRMGNDRVTVLNLLVHKVDAENGVLLIKGAVPGRTGGLVMVRSAIKRGEK

(19) United States
(12) Patent Application Publication
(10) Pub. No.: US 2022/0145366 A1
(43) Pub. Date: May 12, 2022

(54) EARLY DETECTION OF DRUG-RESISTANT MYCOBACTERIUM TUBERCULOSIS

(71) Applicant: THE TRANSLATIONAL GENOMICS RESEARCH INSTITUTE, Phoenix, AZ (US)

(72) Inventors: David Engelthaler, Phoenix, AZ (US); Christopher Allender, Phoenix, AZ (US); James Schupp, Phoenix, AZ (US)

(21) Appl. No.: 17/351,172

(22) Filed: Jun. 17, 2021

Related U.S. Application Data
(60) Provisional application No. 63/040,367, filed on Jun. 17, 2020.

(51) Int. Cl. C12Q 1/689; C12Q 1/686

(52) U.S. Cl. CPC C12Q 1/689; C12Q 2600/156; C12Q 2600/16; C12Q 1/686

(57) ABSTRACT

The present invention relates to oligonucleotides, methods, and kits useful for detecting an antibiotic-resistant subpopulation within a heteroresistant population of Mycobacterium tuberculosis in a sample. An amplicon of a target locus is obtained from the sample. The target locus comprises a region of interest which comprises one or more minor variants associated with the antibiotic resistance. The target locus is selected from the group consisting of: pncA, tlyA, gidB, rpsL, gyrB, embB, ahpC promoter, rplC, and combinations thereof. The amplicon is sequenced on a Next Generation Sequencing (NGS) platform. The region of interest is interrogated to detect the one or more minor variants and thus, the antibiotic-resistant subpopulation of Mycobacterium tuberculosis.

### CROSS REFERENCE TO RELATED APPLICATIONS

This application claims the benefit of and priority to U.S. Provisional Application No. 63/040,367, filed Jun. 17, 2020, the contents of which are hereby incorporated by reference in their entirety.

### REFERENCE TO SEQUENCE LISTING SUBMITTED ELECTRONICALLY

The official copy of the sequence listing is submitted electronically via EFS-Web as an ASCII-formatted sequence listing with a file named “91482-175PAT2_ST25.txt” created on Jun. 17, 2021, and having a size of 44,020 bytes, is filed concurrently with the specification. The sequence listing contained in this ASCII-formatted document is part of the specification and is herein incorporated by reference in its entirety.

### TECHNICAL FIELD

The invention relates to oligonucleotides, methods, and kits for the early detection of drug-resistant Mycobacterium tuberculosis in a sample.

### BACKGROUND OF THE INVENTION

Multi drug-resistant (MDR) and extensively drug-resistant Mycobacterium tuberculosis are increasing worldwide. M. tuberculosis does not naturally contain plasmids, and almost all cases of clinical drug-resistance are caused by single-nucleotide polymorphisms (SNPs) or small insertions/deletions in relevant genes. Heteroresistance, the simultaneous occurrence of drug-resistant subpopulations in an otherwise drug-susceptible bacterial population in a patient, has created uncertainty in the treatment and diagnosis of tuberculosis and is thought to be an important driver of multi-drug resistance in Mycobacterium tuberculosis.

Tuberculosis heteroresistance is not a rare phenomenon, occurring in 9-30% of Mycobacterium tuberculosis populations studied, and has been identified in Mycobacterium tuberculosis populations with phenotypic resistance to first line-drugs (INH, RIF, ETH, and STR) and second-line fluoroquinolones (ofloxacin-OFX) and injectables (AMK). It is highly likely that drug-resistant organisms are present in most tuberculosis lesions, even as very minor population components, given the high bacilli loads that are typically found in patients.

There is a need for methods, primers, and kits useful for rapid, molecular, and phenotypic susceptibility assays to identify and/or quantify M. tuberculosis susceptible or resistant to a drug. In particular, there is a need for compositions and methods useful for detecting and/or quantifying minor resistance variant subpopulations in clinical samples early in therapy to allow for effective treatment of tuberculosis.

### SUMMARY

The present invention is directed to a method of detecting a heteroresistant population of Mycobacterium tuberculosis in a sample, the method comprising: a) providing a sample comprising a population of M. tuberculosis; b) extracting nucleic acids from the sample; c) amplifying a target locus of the genome M. tuberculosis in the extracted nucleic acids, wherein the target locus comprises at least one minor variant associated with drug resistance in M. tuberculosis; d) consecutively sequencing both overlapping nucleic acid strands from a single DNA molecule amplified from the target locus on a Next Generation Sequencing (NGS) platform; e) applying an alignment algorithm to sequencing data from the overlapping nucleic acid strands; and f) performing an analysis of the aligned sequencing data to detect the at least one minor variant and heteroresistant population of the M. tuberculosis In these embodiments, the at least one minor variant may be located within a genomic sequence selected from the group consisting of, but not limited to pncA, tlyA, gidB, rpsL, gyrB, embB, ahpC promoter, and rplC.

In certain implementations, the analysis of the aligned sequencing data is a minor variant analysis. In certain aspects, the minor variant analysis is a haplotype variant analysis. The target locus may be amplified with a high-fidelity polymerase such as KAPA HiFi™ DNA polymerase or Q5® HIFI DNA polymerase.

Typically, each of the overlapping nucleic acid strands consists of less than about 500 nucleotides, less than about 450 nucleotides, less than about 400 nucleotides, less than about 350 nucleotides, less than about 300 nucleotides, less than about 250 nucleotides, less than about 200 nucleotides, less than about 150 nucleotides, less than about 100 nucleotides, or less than about 50 nucleotides.

In other non-limiting aspects, the alignment algorithm is optimized for short nucleotide space reads of less than about 500 nucleotides, less than about 450 nucleotides, less than about 400 nucleotides, less than about 350 nucleotides, less than about 300 nucleotides, less than about 250 nucleotides, less than about 200 nucleotides, less than about 150 nucleotides, less than about 100 nucleotides, or less than about 50 nucleotides. The alignment algorithm may be Novoalign. Various NGS platforms may be used with the present invention including the Ilumina MiSeq platform.

The minor variant analysis may be performed with a bioinformatics script that requires a user to input genomic regions of interest and generates a report with single molecule-overlapping read information used to identify the minor variant.

In yet other aspects, the methods further comprise using a highly homogenous synthetic plasmid standard to identify actual sequence error rate variance between target loci and sequencing runs.

In some embodiments, the minor variant is selected from the group consisting of a single nucleotide polymorphism (SNP), an insertion, and a deletion.

The present invention is also directed to methods of treating a subject in need thereof with a therapeutic agent to a heteroresistant population of M. tuberculosis, wherein the therapeutic agent is selected from the group consisting of PA-824, OPC-67683, SQ109, TMC207, NAS-21, NAS-91, and combinations thereof. In certain aspects, the treatment is preceded by the detection of one or more heteroresistant population of M. tuberculosis in a sample from the subject.

In some aspects, the present invention relates to a method of detecting and/or quantifying a drug-resistant subpopulation of Mycobacterium tuberculosis in a sample, comprising: obtaining an amplicon from the sample, wherein the amplicon comprises a region of interest in pncA (position 2289241 to 2288681 of NC_000962.3), tlyA (position 1917940 to 1918746 of NC_000962.3), gidB (position 4407528 to 4408202 of NC_000962.3), rpsL (position 781560 to 781934 of NC_000962.3), gyrB (position 5240 to 7267 of NC_000962.3), embB (position 4246514 to 4249810 of NC_000962.3), ahpC promoter (position 2726193 to 2726780 of NC_000962.3), rplC (position 800809 to 801462 of NC_000962.3), or a combination thereof, and the region of interest comprises a polymorphism associated with the drug-resistant subpopulation; obtaining sequencing data by sequencing the amplicon on a Next Generation Sequencing (NGS) platform; and detecting and/or quantifying a minor variant of the polymorphism, wherein the presence of the minor variant indicates the presence of the drug-resistant subpopulation.

In one aspect, the subpopulation of Mycobacterium tuberculosis is resistant to pyrazinamide, capreomycin, streptomycin, quinolones, ethambutol, isoniazid, linezolid, or a combination thereof. In another aspect, the subpopulation of Mycobacterium tuberculosis is resistant to streptomycin, ethambutol, linezolid, or a combination thereof.

In certain aspects, obtaining the amplicon uses a primer comprising a sequence at least 85% identical to an oligonucleotide selected from the group consisting of SEQ ID NOs: 1-43 or a complement thereof.

In one aspect, the region of interest comprises a polymorphism in pncA (position 2289241 to 2288681 of NC_000962.3 or SEQ ID NO: 48) associated with the pyrazinamide-resistant subpopulation, and the nucleotide is selected from the group consisting of SEQ ID NOs: 18-21. In certain aspects, the minor variant comprises a deletion of 5′ GCACCC 3′, a deletion of 5′ GGGTGC 3′, or both. In other aspects, the minor variant comprises a deletion of 5′ CGACCC 3′, a deletion of 5′ GGGTGC 3′, or both.

In another aspect, the region of interest comprises a polymorphism in tlyA (position 1917940 to 1918746 of NC_000962.3 or SEQ ID NO: 49) associated with the capreomycin-resistant subpopulation. In one aspect, the oligonucleotide is selected from the group consisting of SEQ ID NOs: 1-6.

In other aspects, the region of interest comprises a polymorphism in gidB (position 4407528 to 4408202 of NC_000962.3 or SEQ ID NO: 50) associated with the streptomycin-resistant subpopulation. In certain aspects, the oligonucleotide is selected from the group consisting of SEQ ID NOs: 7-10.

In yet other aspects, the region of interest comprises a polymorphism in rpsL (position 781560 to 781934 of NC_000962.3 or SEQ ID NO: 51) associated with the streptomycin-resistant subpopulation. In one aspect, the oligonucleotide is selected from the group consisting of SEQ ID NOs: 11-14.

In certain aspects, the region of interest comprises a polymorphism in gyrB (position 5240 to 7267 of NC_000962.3 or SEQ ID NO: 52) associated with the quinolones-resistant subpopulation. In one aspect, the oligonucleotide is selected from the group consisting of SEQ ID NOs: 15-17.

In some aspects, the region of interest comprises a polymorphism in pncA (position 2289241 to 2288681 of NC_000962.3 or SEQ ID NO: 48) associated with the pyrazinamide-resistant subpopulation, and the oligonucleotide is selected from the group consisting of SEQ ID NOs: 22-29.

In one aspect, the region of interest comprises a polymorphism in embB (position 4246514 to 4249810 of NC_000962.3 or SEQ ID NO: 53) associated with the ethambutol-resistant subpopulation. The oligonucleotide may be selected from the group consisting of SEQ ID NOs: 30-33.

In another aspect, the region of interest comprises a polymorphism in ahpC promoter associated with the isoniazid-resistant subpopulation. The oligonucleotide may be selected from the group consisting of SEQ ID NOs: 34-39.

In some aspects, the region of interest comprises a polymorphism in rplC (position 800809 to 801462 of NC_000962.3 or SEQ ID NO: 54) associated with the linezolid-resistant subpopulation. In one aspect, the oligonucleotide is selected from the group consisting of SEQ ID NOs: 40-47.

In other aspects, the method further comprises aligning the sequencing data using an alignment algorithm and interrogating the aligned sequencing data to detect and/or quantify the minor variant of the polymorphism.

In certain aspects, obtaining the sequencing data comprises sequencing complete overlapping complementary strands of the region of interest of each amplicon to obtain independent paired-end reads of the minor variant and calling the minor variant only when the independent paired-end reads of the minor variant are identical.

In one aspect, the sample is selected from the group consisting of: sputum, pleural fluid, blood, saliva, and combinations thereof from a subject.

In some aspects, the disclosed methods further comprise predicting phenotypic Mycobacterium tuberculosis resistance to fluoroquinolones, aminoglycosides, or both based on a micro-heteroresistance threshold. In one aspect, the micro-heteroresistance threshold is about 5.0%, about 4.0%, about 3.0%, about 2.0%, about 1.0%, or about 0.5%.

In some aspects, further comprising administering to the subject a therapeutic agent based on the drug resistance of the Mycobacterium tuberculosis subpopulation in the sample. In one aspect, the therapeutic agent is selected from the group consisting of: an antibiotic, PA-824, OPC-67683, SQ109, TMC207, NAS-21, NAS-91, and combinations thereof. The antibiotic may be any one of Isoniazid, Rifampin (Rifadin, Rimactane), Ethambutol (Myambutol), Pyrazinamide, Bedaquiline (Sirturo), Linezolid (Zyvox), Isonicotinyl Hydrazine, Rifampicin, Ethambutol, PyraZinamide, Moxifloxacin, Cycloserine, Ethambutol, Delamanid, Pyrazinamide, Imipenem-Cilastatin/Meropenem, Amikacin/Streptomycin, Ethionamide/Prothionamide, p-Aminosalicylic Acid, or any combination thereof.

In certain aspects, the present invention relates to a primer for detecting and/or quantifying a drug-resistant subpopulation of Mycobacterium tuberculosis in a sample, comprising a sequence at least 85% identical to an oligonucleotide selected from the group consisting of SEQ ID NOs: 1-47 or a complement thereof, and a label or a modified nucleotide.

In other aspects, the present invention relates to a kit for detecting and/or quantifying a drug-resistant subpopulation of Mycobacterium tuberculosis in a sample, comprising: a primer having a sequence at least 85% identical to an oligonucleotide selected from the group consisting of SEQ ID NOs: 1-47 or a complement thereof, and a label or a modified nucleotide; and reagents for amplification of a genomic sample.

In some aspects, the kit further comprises a fluorescently detectably labeled probe optionally having at least one modified nucleotide, at least one donor fluorescent moiety and at least one corresponding acceptor moiety.

In one aspect, the drug-resistant subpopulation is resistant to capreomycin, and the oligonucleotide is selected from the group consisting of SEQ ID NOs: 1-6.

In another aspect, the drug-resistant subpopulation is resistant to streptomycin, and the oligonucleotide is selected from the group consisting of SEQ ID NOs: 7-14.

In another aspect, the drug-resistant subpopulation is resistant to quinolones, and the oligonucleotide is selected from the group consisting of SEQ ID NOs: 15-17.

In another aspect, the drug-resistant subpopulation is resistant to pyrazinamide, and the oligonucleotide is selected from the group consisting of SEQ ID NOs: 18-29.

In another aspect, the drug-resistant subpopulation is resistant to ethambutol, and the oligonucleotide is selected from the group consisting of SEQ ID NOs: 30-33.

In another aspect, the drug-resistant subpopulation is resistant to isoniazid, and the oligonucleotide is selected from the group consisting of SEQ ID NOs: 34-39.

In another aspect, the drug-resistant subpopulation is resistant to linezolid, and the oligonucleotide is selected from the group consisting of SEQ ID NOs: 40-47.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B depict the cross-sectional relationship between heteroresistant genotype and phenotypic drug susceptibility at individual time points. Beeswarm plots of individual patient isolates demonstrate rrs1401G (FIG. 1A) and gyrase resistance-associated variants (FIG. 1B) stratified by amikacin and ofloxacin phenotypic resistance, respectively. Colors represent SMOR-determined percent mutant, as per legend. The shape of the icon in FIG. 1B denotes mutation type: Circle=high level RAV*; Triangle=low level RAV; diamond=no RAV detected. OFX. The solid black line denotes the ˜20% mutant threshold for >95% PPV derived from ROC analysis. High-level RAVs include gyrA94AAC, 94CAC, 94GGC, 94TAC; all gyrA88 mutations; and gyrB 496CTC, 500CAC; low-level RAVs include gyrA90GTG, 91CCG, 94GCC.

FIG. 2 depicts amikacin pre-resistant signal identification prior to phenotypic resistance. SMOR-determined “pre-resistance” noted a mean of 8.4 months (95% CI, 2.2-14.3 months) prior to phenotypically-determined amikacin resistance (i.e., month=0) in patient isolates. The y-axis displays percent rrs 1401G resistance mutation; the x-axis displays months prior to phenotypic resistance detection. The inset provides zoom on months 7-18 prior to phenotypic resistance with a scale of 0-3% micro-heteroresistance.

FIGS. 3A-3N depict time series graphs of the evolution of fluoroquinolone resistance during treatment. The top row shows four patients that exhibited sub-phenotypic micro-heteroresistant minor populations (<5%) detectable prior to macro-heteroresistance (>5%) at the same QRDR loci. The first four panels of the middle row show patients that exhibited micro-heteroresistant minor populations detectable prior to macro-heteroresistance at different loci. The fifth panel shows a patient that exhibited micro-heteroresistance that was also phenotypically resistant. The bottom row shows five patients that did not exhibit detectable heteroresistance prior to the first time point of macro or fixed resistance detection. Not shown are patients lacking any detectable FQ resistance or lacking the presence of resistance in more than one sampling period. All panels have % mutant plotted on the y-axis and sampling time points on the x-axis, with the initial sample on the left-hand side to the final sample on the right-hand side. Color corresponds to the percent of each resistance loci in the QRDR as described in the legend (upper right corner). Heterogeneity of resistance mutations in multiple patients can be seen by multiple colors at the same time point.

### DETAILED DESCRIPTION

Aspects and applications of the invention presented herein are described below in the detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. The full scope of the inventions is not limited to the specific examples that are described below.

As used herein, the verb “comprise” as is used in this description and the claims and its conjugations are used in its non-limiting sense to mean that items following the word are included, but items not specifically mentioned are not excluded. Also, a reference to an element by the indefinite article “a” or “an” does not exclude the possibility that more than one of the elements are present unless the context clearly requires that there is one and only one of the elements. The indefinite article “a” or “an” thus usually means “at least one”.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the term “detecting” refers to determining the presence or absence of.

As used herein, the term “quantitating” refers to determining the specific amount or ratio of.

As used herein, the term “sample” refers to a sample of biological tissue or fluid that comprises nucleic acids. Such samples include, but are not limited to, tissue isolated from animals. Samples may also include sections of tissues such as biopsy and autopsy samples, frozen sections taken for histologic purposes, blood, plasma, serum, sputum, saliva, stool, tears, mucus, hair, and skin. A sample may be provided by removing a sample of cells from an animal but can also be accomplished by using previously isolated cells (e.g., isolated by another person, at another time, and/or for another purpose). Such samples may also include all clinical samples including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates.

As used herein, the term “antibiotic” refers to a drug (medicine) that inhibits the growth of or destroys M. tuberculosis.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-O-methyl Ribo-U, 2′-O-methyl Ribo-C, an N4-ethyl-dC, an N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference.

The term “complement thereof” refers to nucleic acid that is both the same length as, and exactly complementary to, a given nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

As used herein, the term “amplicon” refers to a piece of DNA or RNA that is the source and/or product of amplification or replication events.

As used herein, the term “target locus” refers to a fixed position on a Mycobacterium tuberculosis chromosome, such as the position of a gene or a marker.

As used herein, the term “drug resistance” or “antibiotic resistance” refers to the ability of M. tuberculosis to resist the effects of an antibiotic.

As used herein, the term “multiple drug resistance,” “multidrug resistance,” “multiresistance,” or MDR refers to antimicrobial resistance by a population or subpopulation of Mycobacterium tuberculosis to multiple antimicrobial drugs, e.g., multiple antibiotics.

As used herein, the term “extensively drug-resistant TB (XDR TB)” refers to a type of multidrug-resistant tuberculosis (MDR TB) that is resistant to isoniazid and rifampin, plus any fluoroquinolone and at least one of three injectable second-line drugs (i.e., amikacin, kanamycin, or capreomycin).

As used herein, the term “region of interest” refers to contiguous or noncontiguous DNA sequence of the target locus identified for a particular purpose. In some aspects, the region of interest refers to a contiguous region of at least 10 nucleotides and less than 500 nucleotides.

As used herein, the term “variant” “genetic variant” refers to a specific region of the genome which differs between two Mycobacterium tuberculosis genomes. Non-limiting examples include a single-nucleotide polymorphism (SNP), or a mutation, such as an insertion or a deletion. The minor variant detected in the heteroresistant population of M. tuberculosis may be an SNP, an insertion, or a deletion. Non-limiting examples of genetic mutations associated with drug resistance in M. tuberculosis are found in Georghiou et al. (2012) PLoS ONE 7(3):e33275.

As used herein, the term “single-nucleotide polymorphism” of “SNP” refers to a substitution of a single nucleotide that occurs at a specific position in the genome, where each variation is present to some appreciable degree within a population (e.g., >1%).

As used herein, the term “subpopulation” refers to an identifiable fraction or subdivision of a population.

As used herein, the term “read” or “sequence read” refers to the sequence of a cluster that is obtained after the end of the sequencing process which is ultimately the sequence of a section of a unique fragment.

As used herein, the term “fixed resistance” refers to the presence of at least 95% resistant dominant population in an individual sample.

As used herein, the term “macro-heteroresistance” refers to the presence of between least 5% to less than 95% resistant subpopulations in an individual sample.

As used herein, the term “micro-heteroresistance” refers to the presence of greater than 0.1% to less than 5% resistant subpopulations in an individual sample, as defined previously³⁹.

As used herein, the term “pre-resistance” describes samples that have a heteroresistant genotype with susceptible phenotype and subsequently progress to increased levels of genomic heteroresistance or fixed resistance while also attaining phenotypic resistance.

In some embodiments, the present invention is directed to a next-generation sequencing analysis methodology to detect minor proportions of a sample that contain mutations associated with important phenotypes, including antibacterial resistance. This analysis decreases the sequencing error rate so that extremely low levels of true minor components (e.g., SNP loci) can be detected.

The incidence of drug-resistant (DR) tuberculosis (TB) continues to increase worldwide. Undetected heteroresistance, the presence of DR and susceptible genotypes in bacterial populations involved in infection, at treatment initiation may play a role in the expansion of DR strains and treatment failure. In Mycobacterium tuberculosis (Mtb), current minor DR component detection levels are limited to −1%, using phenotypic drug susceptibility testing, which requires 15-30 days or even longer to complete. By that point during an infection, it is likely too late to prevent DR-TB and treatment failure.

In some aspects, the present invention relates to a method of detecting resistant Mtb sub-populations consisting of 0.1% or less of the total Mtb population in under a week. Detection of minor components in complex biological mixtures has radically advanced with the emergence of next-generation sequencing. Low-level detection from sequence data, however, is not trivial, primarily due to the error rates in sequencing. The error associated with the respective sequencing platform, as well as the GC content of the organism, sets the limit of discerning actual minor component from error. However, the use of “single molecule-overlapping reads” (SMOR) analysis for determination of actual mutation ratios in target loci (e.g., antibiotic resistance genes) leads to an increase in heteroresistance detection sensitivity and lower error bias.

The use of overlapping reads allows for effective coverage of each locus on both strands of an individual sequenced DNA molecule, which in turn allows for an independent confirmation of the specific nucleotide at that single locus. The product rule of probability applies, such that if one locus on a single molecule is read two times, it has the lower limit of detection of the probability of one error occurring squared. In some embodiments, the Illumina Miseq platform is used to sequence amplicons from several different in vitro mixtures of DR and susceptible Mtb strains to validate the use of SMOR for identifying heteroresistance. The calculated average of combined amplification and sequencing error rate for Mtb (a high GC organism) is 0.51% per position across the amplicons tested. When employing SMOR, the theoretical limit of detection of a minor component is 2.6×10⁻⁶, readily allowing for detection of minor components below 0.51%.

The Inventors have been able to detect a 0.3% artificial mixture of SNP alleles in the inhA promoter at a frequency of 3.07×10⁻³, which was at least two orders of magnitude more frequent than identifiable sequence errors. The use of SMOR allows for researchers and clinicians to follow the evolution of heteroresistance, determine its clinical relevance and develop appropriate treatment strategies to suppress minor component resistant sub-populations before they become clinically significant.

In Mycobacteria tuberculosis (Mtb) there are characterized SNPs that confer resistance to several different antibiotics. By using overlapping reads on these targeted regions, we can characterize heteroresistance in clinical samples down to a level that has not been previously achieved. Overlapping reads have been used in next generation sequencing to improve whole genome examination but they have not been used to add confidence in antibiotic resistance population evaluation.

With the invention, clinicians are able to track patient treatment in a more timely fashion and alter the course of treatment when heteroresistance is detected within a week versus a month or more as is common with current technology. This analysis can also be useful to researchers wanting to characterize population structure within a single sample of bacteria.

In one embodiment, the invention provides a diagnostic assay for the detection of heteroresistance in Mycobacterium tuberculosis in clinical samples.

In some embodiments, the limit of detection for the minor variant is less than about 1.0%, less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, or less than about 0.01% of the heteroresistant population.

In other embodiments, a micro-heteroresistance threshold is determined to predict phenotypic Mycobacterium tuberculosis resistance to pyrazinamide, capreomycin, streptomycin, quinolones, ethambutol, isoniazid, linezolid, or a combination thereof. In some embodiments the micro-heteroresistance threshold is less than about 5.0%, less than about 4.0%, less than about 3.0%, less than about 2.0%, less than about 1.0%, less than about 0.9%, less than about 0.8%, less than about 0.7%, less than about 0.6%, less than about 0.5%, less than about 0.4%, less than about 0.3%, less than about 0.2%, or greater than about 0.1% of the population.

In other embodiments, each of the overlapping nucleic acid strands to be sequenced with the disclosed method consists of less than about 500 nucleotides, less than about 450 nucleotides, less than about 400 nucleotides, less than about 350 nucleotides, less than about 300 nucleotides, less than about 250 nucleotides, less than about 200 nucleotides, less than about 150 nucleotides, less than about 100 nucleotides, or less than about 50 nucleotides.

In yet other embodiments, the disclosed method further comprises administering a therapeutic agent to a heteroresistant population of M. tuberculosis. Exemplary therapeutic agents are found in Da Silva et al. (2011) J. Antimicrob. Chemother. 66:1417.

The minor variant detected in the heteroresistant population of M. tuberculosis may be an SNP, an insertion, or a deletion. Non-limiting examples of genetic mutations associated with drug resistance in M. tuberculosis are found in Georghiou et al. (2012) PLoS ONE 7(3):e33275.

Mathematical models of within-host Mtb population dynamics have predicted that heteroresistance can cause the emergence of MDR-TB prior to treatment initiation, and this emergence may occur 1,000-10,000 times more frequently. Studies of within-host dynamics of Mtb growth during treatment of have also indicated that resistant subpopulations can easily dominate a lesion over time in both treatment compliant and non-compliant patients. The presence of resistance conferring mutations, even as minor components of an infecting population of Mtb, likely leads to selection of resistant strains, in the presence of the corresponding drug, and subsequent treatment failure. Minor resistant populations, however, are typically missed through standard analysis of isolates because the dominant organism phenotype masks any minor component variants. In certain aspects, the present invention addresses this problem by providing effective methods to detect and quantify minor resistant populations.

In some embodiments, the present invention is directed to the detection and analysis of heteroresistance in tuberculosis infections. An assay is provided that is able to accurately detect heteroresistance in Mtb and quantify the presence and proportion of all resistant allele minor components down to less than 0.1% using clinically relevant table-top next generation sequencing (NGS) technology and advanced bioinformatic algorithms. This approach provides a rapid, highly sensitive and specific method for detecting and monitoring the potential clinical relevance of heteroresistance in serial clinical samples from TB patients, which is not achievable by any other existing technology. Additionally, the NGS technology used in the assay can be used for deep sequencing of multiple targeted areas simultaneously, which allows for the detection of extremely rare minor components in a population at all targeted locations at once. This multiplexing approach is ideal for developing a practical, efficient and rapid analysis of heteroresistance directly from patient sputum, which has significant advantages over existing technologies.

While deep-sequencing seems to be an obvious solution, it is not sufficient, in and of itself. NGS minor variant detection is not trivial; primarily due to the error rates associated with the sequencing platform (e.g. Illumina MiSeq platform has a standard rating of 75% of bases having a 0.1% error). This rate sets a theoretical limit of discerning a rare variant from error but recent advances in technology and bioinformatics allow for minor variant detection at significantly lower levels than expected error rate. An advantage resulting from the approach of the present invention is the ability to accurately detect minor components below the sequencing error by using a “Single-Molecule Overlapping Read” (SMOR) analysis.

In certain aspects, the present invention relates to an approach to applying cutting-edge genomic science and technology to the ongoing clinical and public health problem of multi-drug resistant tuberculosis. In one embodiment, an optimized heteroresistance assay is used to detect known mutations associated with seven anti-TB drugs, followed by an evaluation of heteroresistance in serial samples from a patient population.

In some embodiments, the present invention further comprises administering to the subject a regime of antibiotics to effectively control the population of pathogen based on the presence or absence of antibiotic resistance markers in the pathogen.

In certain aspects, the present invention is used to detect and monitor antibiotic resistance in a subject infected with Mycobacterium tuberculosis. Antibiotic resistance can be determined by the presence or absence of one or more antibiotic resistance genes or markers in the population. Non-limiting examples of such antibiotic resistance genes include pncA, tlyA, gidB, rpsL, gyrB, embB, ahpC promoter, and rplC.

In certain embodiments, the method of the present invention further comprises treating the subject with an antibiotic or regime of antibiotics. Non-limiting examples of such antibiotics include PA-824, OPC-67683, SQ109, TMC207, NAS-21, NAS-91, and combinations thereof.

In some embodiments, the nucleic acids from the sample are analyzed by Sequencing by Synthesis (SBS) techniques. SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in some of the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides. In methods using nucleotide monomers lacking terminators, the number of different nucleotides added in each cycle can be dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.). In preferred methods a terminator moiety can be reversibly terminating.

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.). However, it is also possible to use the same label for the two or more different nucleotides present in a sequencing reagent or to use detection optics that do not necessarily distinguish the different labels. Thus, in a doublet sequencing reagent having a mixture of A/C both the A and C can be labeled with the same fluorophore. Furthermore, when doublet delivery methods are used all of the different nucleotide monomers can have the same label or different labels can be used, for example, to distinguish one mixture of different nucleotide monomers from a second mixture of nucleotide monomers. For example, using the [First delivery nucleotide monomers]+[Second delivery nucleotide monomers] nomenclature set forth above and taking an example of A/C+(1/T), the A and C monomers can have the same first label and the G and T monomers can have the same second label, wherein the first label is different from the second label. Alternatively, the first label can be the same as the second label and incorporation events of the first delivery can be distinguished from incorporation events of the second delivery based on the temporal separation of cycles in an SBS protocol. Accordingly, a low resolution sequence representation obtained from such mixtures will be degenerate for two pairs of nucleotides (T/G, which is complementary to A and C, respectively; and C/A which is complementary to G/T, respectively).

Some embodiments include pyrosequencing techniques. Pyrosequencing detects the release of inorganic pyrophosphate (Ppi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released Ppi can be detected by being immediately converted to adenosine triphosphate (ATP) by ATP sulfurylase, and the level of ATP generated is detected via luciferase-produced photons.

In another example type of SBS, cycle sequencing is accomplished by stepwise addition of reversible terminator nucleotides containing, for example, a cleavable or photobleachable dye label as described, for example, in U.S. Pat. No. 7,427,67, U.S. Pat. No. 7,414,1163 and U.S. Pat. No. 7,057,026, the disclosures of which are incorporated herein by reference. This approach is being commercialized by Solexa (now Illumina Inc.), and is also described in WO 91/06678 and WO 07/123,744 (filed in the United States Patent and Trademark Office as U.S. Ser. No. 12/295,337), each of which is incorporated herein by reference in their entireties. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In other embodiments, Ion Semiconductor Sequencing is utilized to analyze the nucleic acids from the sample. Ion Semiconductor Sequencing is a method of DNA sequencing based on the detection of hydrogen ions that are released during DNA amplification. This is a method of “sequencing by synthesis,” during which a complementary strand is built based on the sequence of a template strand.

For example, a microwell containing a template DNA strand to be sequenced can be flooded with a single species of deoxyribonucleotide (dNTP). If the introduced dNTP is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple dNTP molecules will be incorporated in a single cycle. This leads to a corresponding number of released hydrogens and a proportionally higher electronic signal.

This technology differs from other sequencing technologies in that no modified nucleotides or optics are used. Ion semiconductor sequencing may also be referred to as ion torrent sequencing, proton-mediated sequencing, silicon sequencing, or semiconductor sequencing. Ion semiconductor sequencing was developed by Ion Torrent Systems Inc. and may be performed using a bench top machine. Rusk, N. (2011). “Torrents of Sequence,” Nat Meth 8(1): 44-44. Although it is not necessary to understand the mechanism of an invention, it is believed that hydrogen ion release occurs during nucleic acid amplification because of the formation of a covalent bond and the release of pyrophosphate and a charged hydrogen ion. Ion semiconductor sequencing exploits these facts by determining if a hydrogen ion is released upon providing a single species of dNTP to the reaction.

For example, microwells on a semiconductor chip that each contain one single-stranded template DNA molecule to be sequenced and one DNA polymerase can be sequentially flooded with unmodified A, C, G or T dNTP. Pennisi, E. (2010). “Semiconductors inspire new sequencing technologies” Science 327(5970): 1190; and Perkel, J., “Making contact with sequencing's fourth generation” Biotechniques (2011). The hydrogen ion that is released in the reaction changes the pH of the solution, which is detected by a hypersensitive ion sensor. The unattached dNTP molecules are washed out before the next cycle when a different dNTP species is introduced.

Beneath the layer of microwells is an ion sensitive layer, below which is a hypersensitive ISFET ion sensor. All layers are contained within a CMOS semiconductor chip, similar to that used in the electronics industry. Each released hydrogen ion triggers the ISFET ion sensor. The series of electrical pulses transmitted from the chip to a computer is translated into a DNA sequence, with no intermediate signal conversion required. Each chip contains an array of microwells with corresponding ISFET detectors. Because nucleotide incorporation events are measured directly by electronics, the use of labeled nucleotides and optical measurements are avoided.

An example of an Ion Semiconductor Sequencing technique suitable for use in the methods of the provided disclosure is Ion Torrent sequencing (U.S. Patent Application Numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H+), which signal detected and recorded in a sequencing instrument. The signal strength is proportional to the number of nucleotides incorporated. User guides describe in detail the Ion Torrent protocol(s) that are suitable for use in methods of the invention, such as Life Technologies' literature entitled “Ion Sequencing Kit for User Guide v. 2.0” for use with their sequencing platform the Personal Genome Machine™ (PCG).

In some embodiments, as a part of the sample preparation process, “barcodes” may be associated with each sample. In this process, short oligos are added to primers, where each different sample uses a different oligo in addition to a primer.

The term “library”, as used herein refers to a library of genome-derived sequences. The library may also have sequences allowing amplification of the “library” by the polymerase chain reaction or other in vitro amplification methods well known to those skilled in the art. The library may also have sequences that are compatible with next-generation high throughput sequencers such as an ion semiconductor sequencing platform.

In certain embodiments, the primers and barcodes are ligated to each sample as part of the library generation process. Thus during the amplification process associated with generating the ion amplicon library, the primer and the short oligo are also amplified. As the association of the barcode is done as part of the library preparation process, it is possible to use more than one library, and thus more than one sample. Synthetic DNA barcodes may be included as part of the primer, where a different synthetic DNA barcode may be used for each library. In some embodiments, different libraries may be mixed as they are introduced to a flow cell, and the identity of each sample may be determined as part of the sequencing process. Sample separation methods can be used in conjunction with sample identifiers. For example, a chip could have 4 separate channels and use 4 different barcodes to allow the simultaneous running of 16 different samples.

The present invention is further illustrated by the following examples that should not be construed as limiting. The contents of all references, patents, and published patent applications cited throughout this application, as well as the Figures, are incorporated herein by reference in their entirety for all purposes.

The following examples are given for purely illustrative and non-limiting purposes of the present invention.

### EXAMPLES

### Example 1. Early, “Pre-Resistant” Genetic Signals Predate the Emergence of Phenotypic Mycobacterium tuberculosis Resistance

Tuberculosis is the leading global cause of infectious disease death, and drug-resistant forms of Mycobacterium tuberculosis (Mtb) are an increasingly ominous public health threat. Heteroresistance in M. tuberculosis is a known driver of acquired drug resistance during therapy. The detection of resistant micro-populations (<5%) in an otherwise dominantly susceptible infection may be an early indicator of an impending multi-drug or extensively-drug resistant outcome. Here we seek to determine the role of micro-heteroresistance in subsequent full resistance and treatment failure.

Incremental gains in global TB control are juxtaposed against an increasing burden of drug resistance¹⁹. Heteroresistance refers to the presence of mixed populations of drug-resistant and drug-sensitive organisms within a specific clinical specimen and is a diagnostic and treatment dilemma in the field of HIV^{20,21}, oncology^{22,23}, and TB^{24,25}, where it has been significantly underreported²⁶⁻³¹. Clinical and epidemiologic studies of M.tb heteroresistance have been complicated by the lack of an ultra-sensitive reference standard. We hypothesized that detectable sub-phenotypic, minor (i.e., <1%) resistant M. tuberculosis variants might precede the development of phenotypic resistance. Their detection could allow crucial alterations in patient management to avert the development of full second-line drug resistance.

Fluoroquinolones and aminoglycosides are of particular interest because they define XDR-TB, the genotypic signatures conferring phenotypic resistance are well-established, and particular resistance-associated variants (RAVs) are associated with mortality³². As next-generation sequencing (NGS) cost and operational complexity continue to decline in clinical settings^{33,34}, differentiation of true RAVs (particularly those occurring at very low frequencies)^{35,36} from sequencing error remains a major barrier to interpretable ultra-deep sequencing of clinical specimens. We have previously developed a novel NGS approach (Single Molecule-Overlapping Reads, or SMOR)^{37,38} that reduces sequencing error (i.e., false-positive calls) in critical drug resistance determining region sequences in M.tb by orders of magnitude, from 1% to approximately 0.01%³⁷. In this approach, NGS amplicon libraries are designed such that there is complete overlap of forward and reverse paired-end reads from the same DNA molecule. This provides two independent base calls at each position within the same DNA fragment, which significantly lowers the probability of an erroneous base call³⁶. SMOR also overcomes metagenomic complexities of clinical sputum specimens³⁸, as it only amplifies pre-determined regions of the M. tuberculosis genome, allowing for high-resolution analysis through the enrichment of select targets. We have recently demonstrated low-level minor resistant populations at levels between 0.1% and 5% (i.e., micro-heteroresistance) using SMOR³⁹.

### Methods

### Routine Drug Susceptibility Testing and DNA Extraction

138 serial sputa from 18 MDR-TB cases were identified, which acquired further phenotypic resistance to fluoroquinolones, aminoglycosides, or both. Sputum specimens were collected from high-risk patients (previously treated for TB, failing first-line therapy or in contact with a patient with drug-resistant TB) or from patients found to be RIF resistant by Gene Xpert (since 2011), in accordance with the national TB control program. Isolates were submitted to the National Health Laboratory Service (NHLS) for drug sensitivity testing (DST) of isoniazid and rifampin using the indirect proportion method on Middlebrooks 7H11 according to the World Health Organization (WHO) recommended critical concentrations. Isolates resistant to isoniazid and rifampicin were subjected to a second round of DST. Briefly, decontaminated and liquefied sputum was cultured in the MGIT 960 (Becton Dickinson, Sparks, Md.) system until positive (by acid-fast bacilli smear and M. tuberculosis speciation), after which DST was done on Middlebrook 7H11 slants (Becton Dickinson, Sparks, Md.) containing 2 μg/ml and 4 μg/ml for ofloxacin and amikacin, respectively. The control slants of all isolates found to be resistant to any drug were submitted to Stellenbosch University (SU) for storage. The isolates were stored at SU by adding a scrape of the M. tuberculosis growth to 400 μl Tween80 saline solution (0.001% Tween80 and 0.08M NaCl). A crude DNA lysate was generated by incubation of half of the cells (200 μl) at 100° C. for 30 min. The other half of the cells were stored on glass beads with proteose peptone growth medium at −80° C. for future use.

### Second-Line Phenotypic DST

Additional second-line phenotypic DST was performed at SU from the stored cultures. DST was performed by indirect proportion method in the BD BACTEC™ MGIT™ 960 system in conjunction with the BD EpiCenter™ system and using a concentration of 4 μg/ml amikacin.

### SMOR Assay

Isolates for which crude M. tuberculosis DNA extracted from the original DST 7H11 agar was available and viable were selected for targeted deep sequencing. DNA specimens were coded, blinded, amplified and prepared for targeted SMOR sequencing, as described previously³⁷, with the following modifications. Following the gene-specific multiplex PCR, primer-dimer artifacts were removed using a single 0.8×, Agencourt AMPure XP bead (Beckman Coulter, Brea, Calif.) purification, instead of two sequential bead purifications, eluting the amplicons in 15 μL of a 10 mM Tris-HCl, 0.05% Tween 20 solution. The SMOR assay's gene-specific multiplex PCR contained gene regions critical for detecting mutations associated with the XDR phenotype: rpoB to characterize RIF resistance; gyrA to characterize FQ resistance; rrs to characterize AMK resistance; and the eis promoter and rrs to characterize kanamycin resistance³⁷. All target alleles were covered with 10 or more SMOR reads (i.e., ≥20 standard reads, a pair of independent reads for each sequenced amplicon molecule), and 58% were covered with 100 or more SMOR reads (≥200 standard reads). Numerous no-template controls were used throughout the prep process to ensure lack of well-to-well sample or amplicon contamination. DNA from a confirmed pan-susceptible M. tuberculosis H37Rv strain was used as a sequencing error control throughout the SMOR assay, as described previously³⁷. All sequencing read files were deposited in the NIH Short Read Archive (Bioproject ID #: PRJNA503635). Primer sequences are shown in Table 2.

### ASAP Sequencing Analysis

The previously published SMOR analysis tool³⁷ was incorporated into the TB Amplicon Sequencing Analysis Pipeline (ASAP) software³⁸. Briefly, this software automates the process of quantifying the alleles of interest within gene regions of interest for every overlapping read pair. Paired reads from the same DNA molecule that disagree invariably indicate sequencing error and were excluded. Therefore, the use of overlapping reads allows for high confidence of low-level subpopulation (>0.1%) detection, well below standard sequencing error rates³⁷. Targeted sequencing additionally allows for the detection of multiple RDR-associated RAVs within individual amplicons (i.e., haplotype analysis). The ASAP software detects and quantifies the presence of multiple RAV haplotypes among the amplicons to further analyze the nature of heteroresistance within resistant subpopulations.

### Receiver Operating Characteristic (ROC) and Statistical Analyses

ROC analysis was conducted to evaluate the presence of micro-heteroresistant subpopulations in patient samples prior to phenotypic transition (i.e., for prediction of patients that move towards fixed resistance). Micro- and macro-resistant designated samples were treated as distinct sub-groups for calculation of assay metrics and also pooled with fixed resistant calls for ROC calculations (Table 1). Because repeated measures of the SMOR percent mutant were used as predictors in the ROC analysis, confidence intervals for the area under the curve were obtained using bootstrap resampling by the participant, and for sensitivity, specificity and PPV using GEE logistic models with robust standard errors. Statistical analysis was conducted in R 3.4.1, Rstudio 1.0.153, Stata Version 15.1, and Microsoft Excel (version 14.7.7).

### Findings

SMOR, a high-resolution targeted sequencing method, was used to detect and quantify critical resistance mutations for the second-line aminoglycosides and fluoroquinolones. DNA from 138 serial primary cultures were collected from 18 MDR-TB patients in the Western Cape, South Africa. The 18 patients, a retrospective cohort, were known to have acquired phenotypic resistance to second-line injectable (SLI) aminoglycosides and/or fluoroquinolones (FQ). They advanced from multi-drug resistant tuberculosis to extensively drug-resistant tuberculosis, while assumed to be under standard therapy.

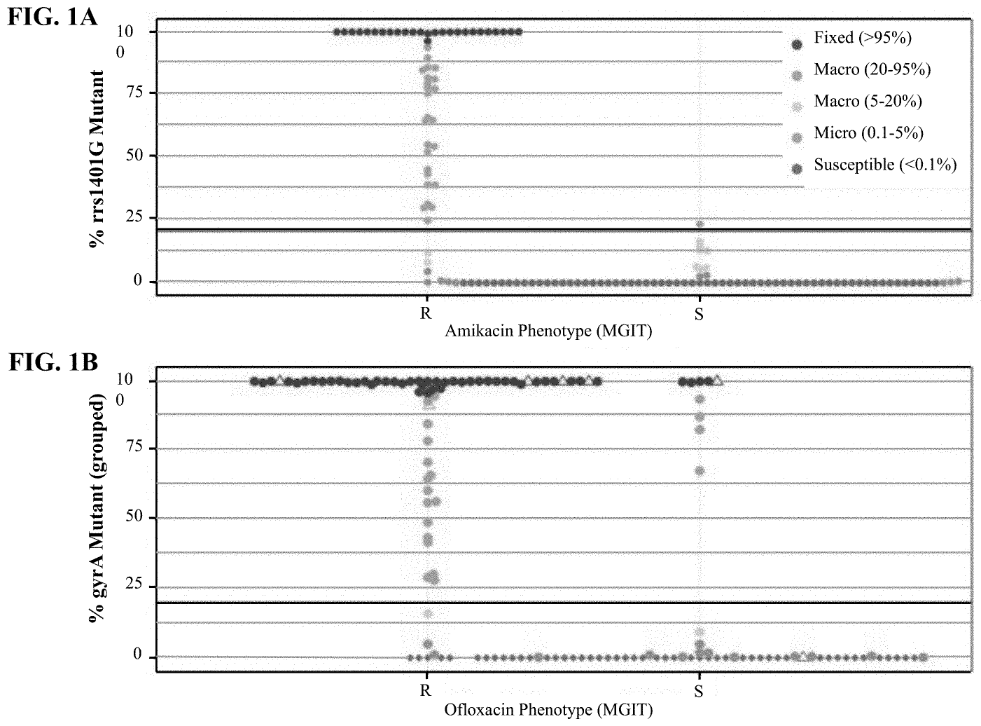

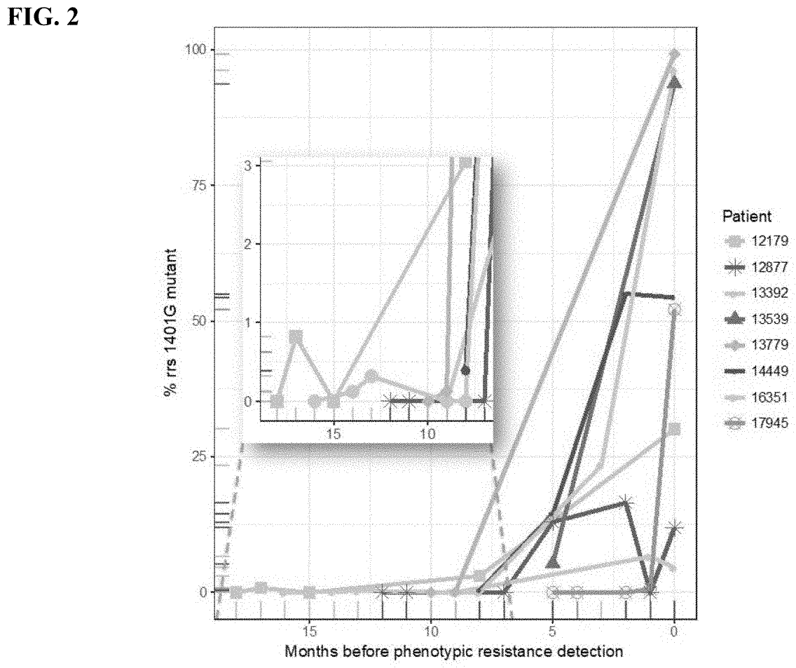

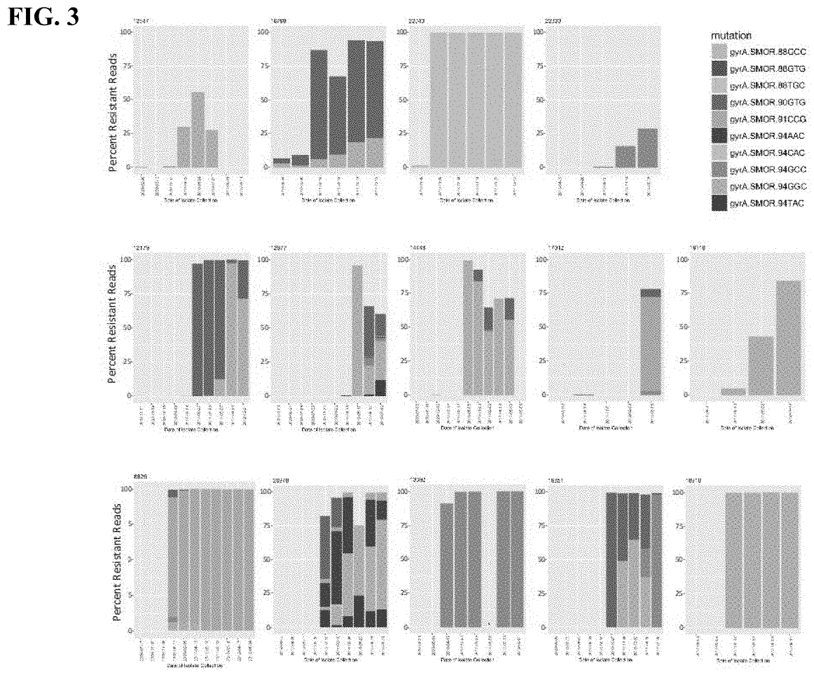

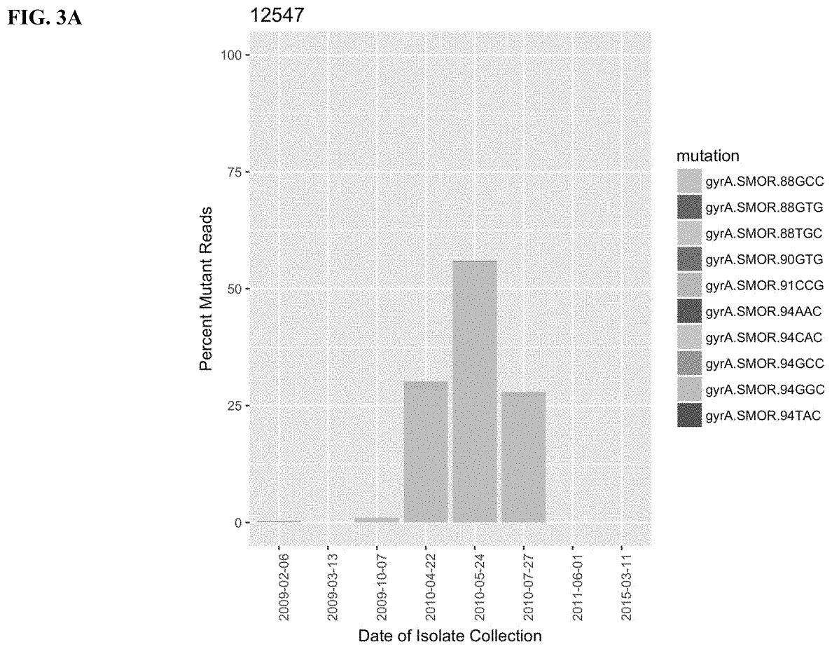

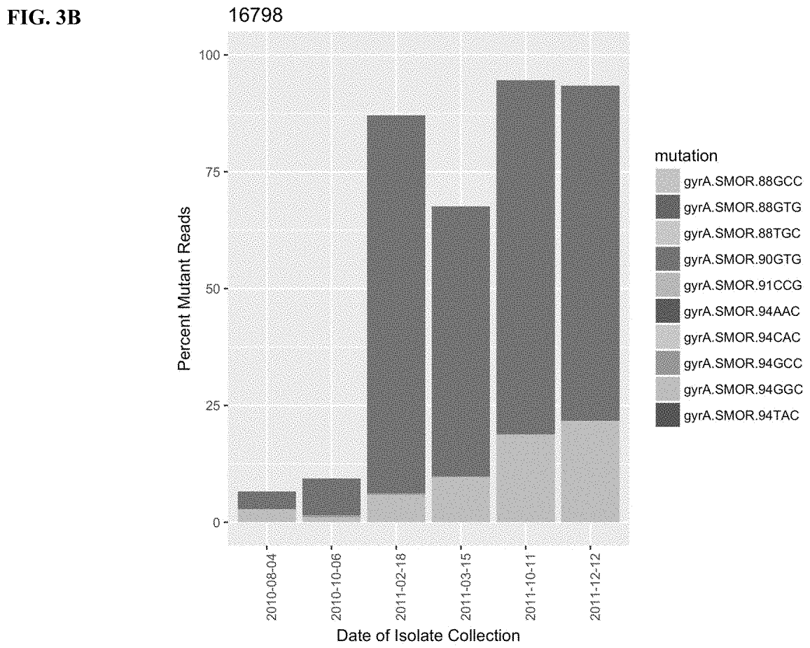

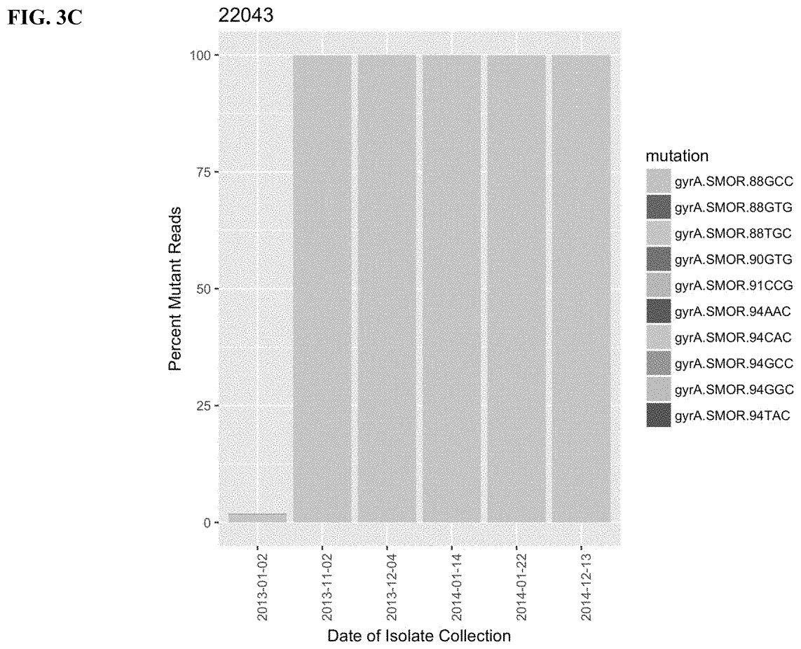

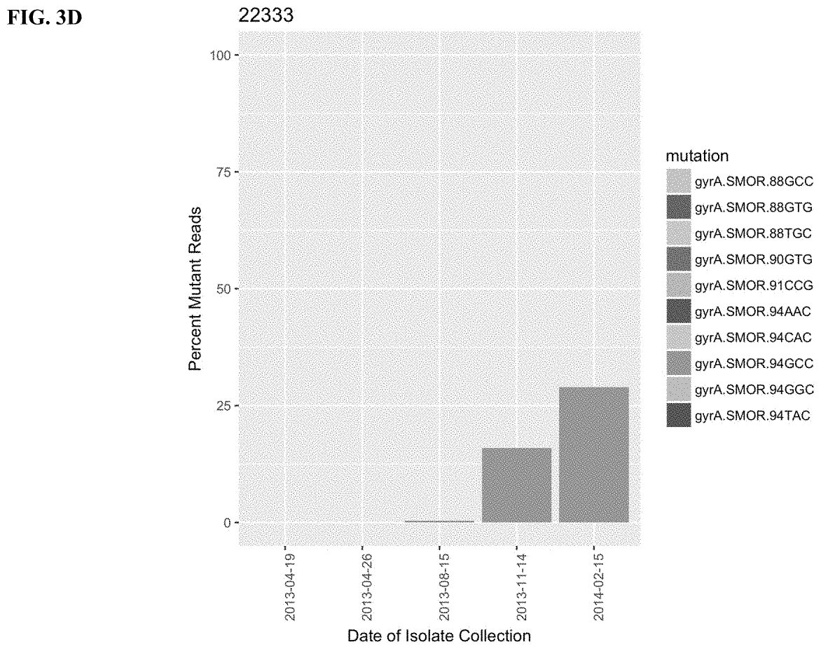

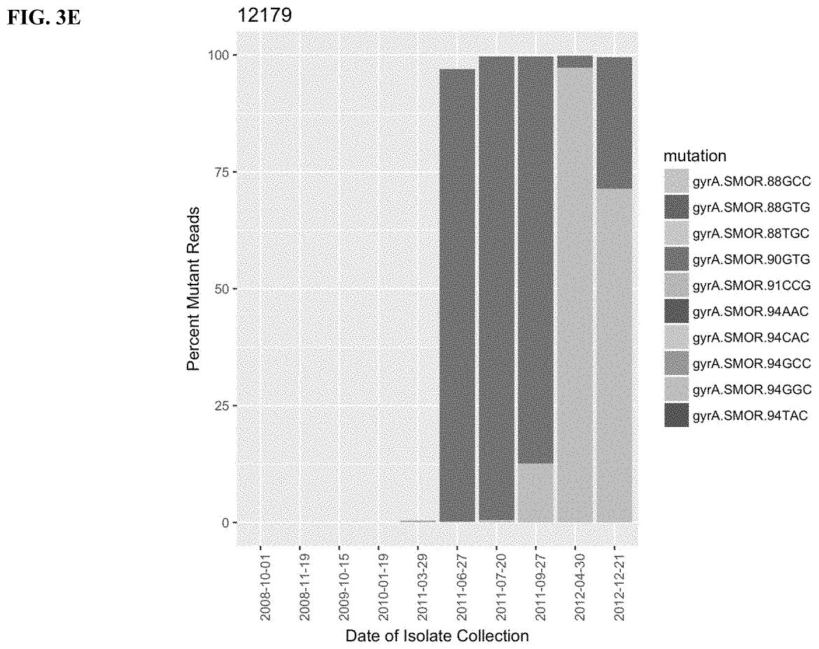

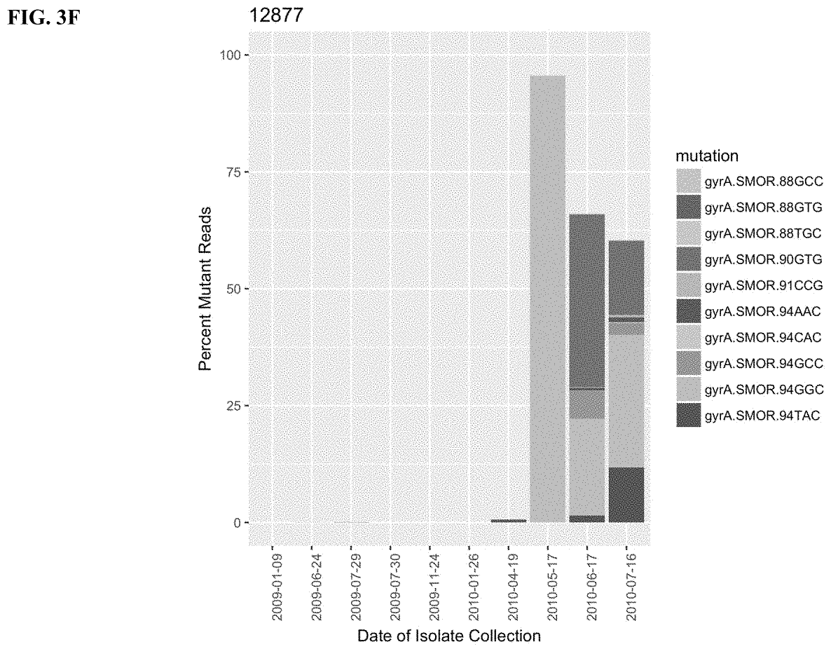

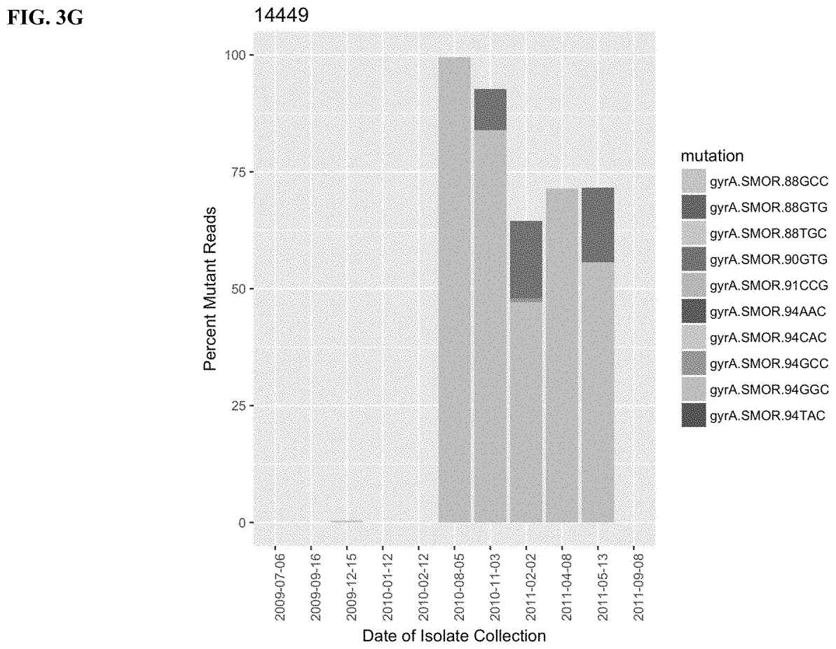

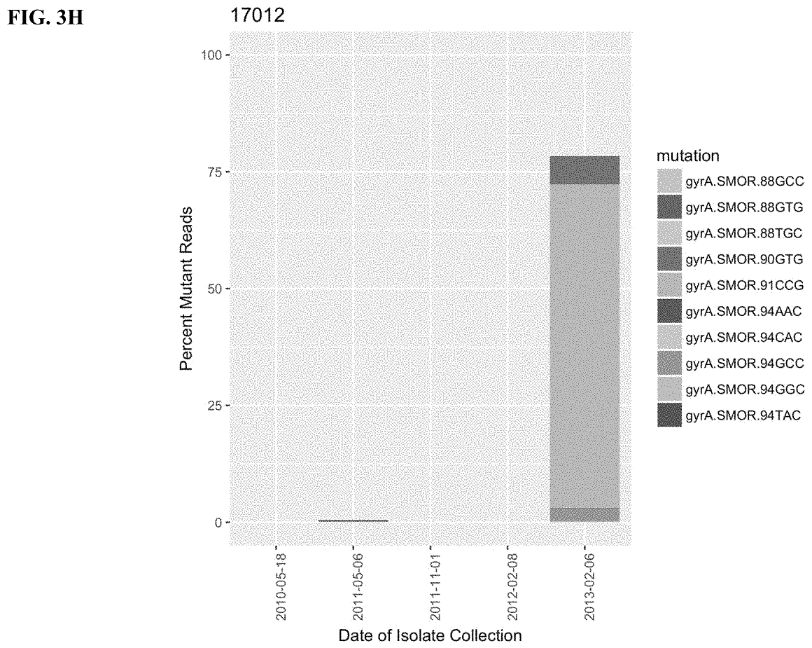

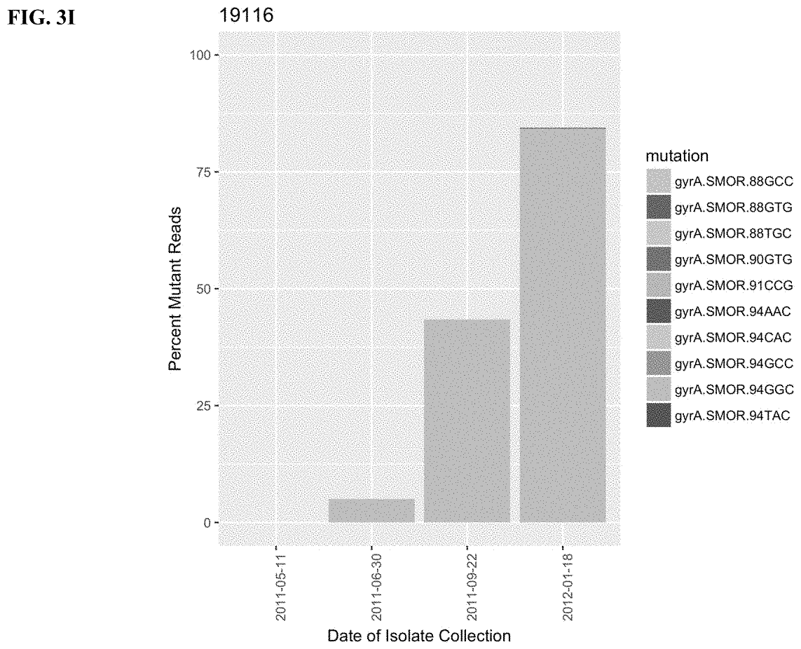

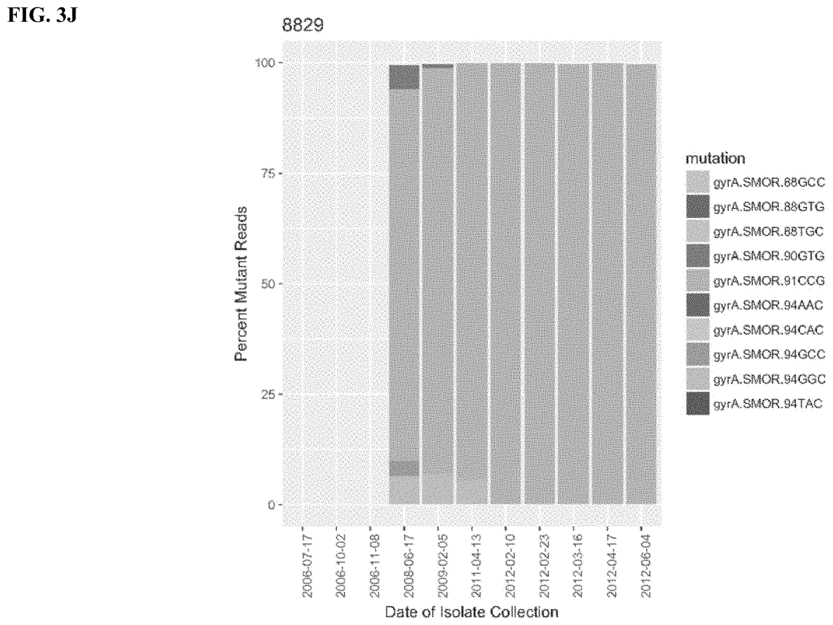

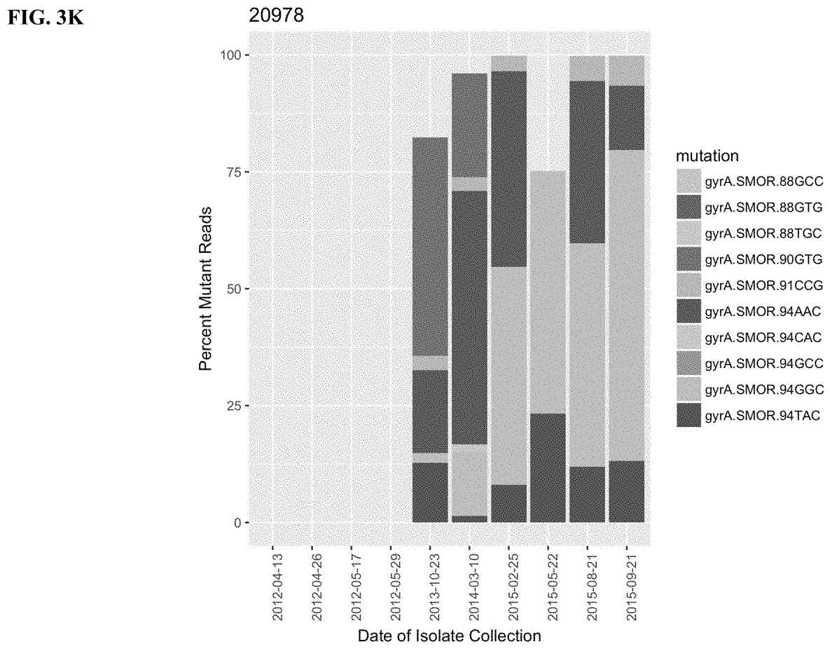

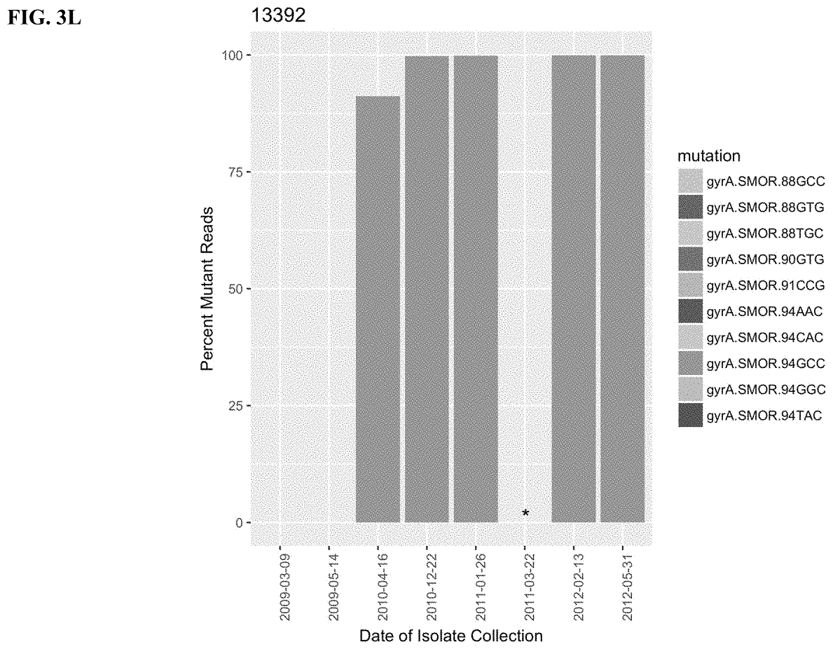

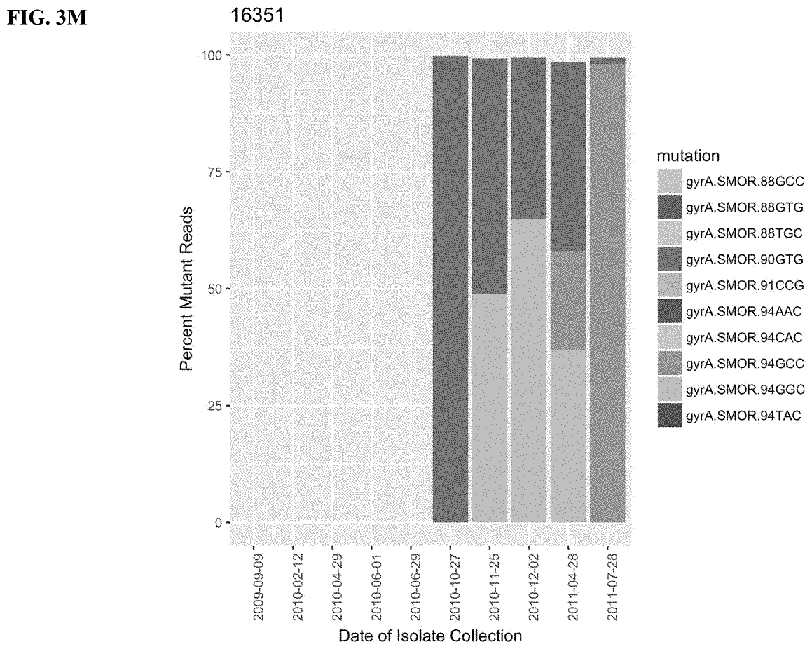

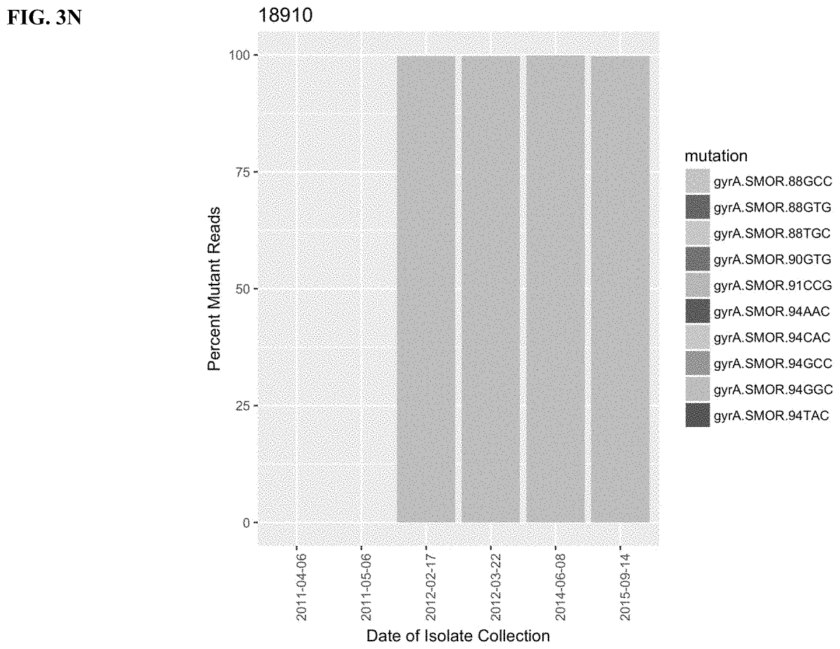

Treatment records were available for eight of these patients. Among them, seven (88%) were treated with contemporary standardized MDR-TB regimens including an FQ and SLI. Resistance determining loci in the rrs and gyrA genes, respectively, were analyzed, and the SMOR percent mutant for each sampling point and patient were used as Receiver Operating Characteristic (ROC) predictors. Sequence analysis identified numerous instances of micro- (<5%) and macro-heteroresistance (>5%) in phenotypically drug-susceptible samples at all time points (FIGS. 1A and 1B), which subsequently increased to fixed resistance (>95%) during the sampling period. Baseline isolates with genotypic micro-heteroresistance and phenotypic drug-susceptibility showed a propensity to increase to fixed genotypic resistance and phenotypic drug resistance that varied by the drug. Receiver operator characteristic (ROC) analysis identified an optimal micro-heteroresistance threshold of 0.12% (98.7% sensitivity (95% CI, 96.1%-100.0%)) (Table 1) for M. tb variants proceeding to full SLI (amikacin) resistance. 100% specific predictive ability for the detection of eventually fixed aminoglycoside resistance when patient samples achieved a micro-heteroresistance threshold of 0.12%. This “pre-resistant” signal for amikacin resistance was observed in eight of the eleven patients that converted from susceptible to resistant, at a mean of 8.4 months (95% CI, 2.2-14.3 months) prior to resistant phenotypic detection (FIG. 2).

The sensitivity of micro-heteroresistance was also high (>90%) for subsequent fluoroquinolone resistance, yet specificity was less than 10%, depending on the resistance loci detected. At similar levels of sub-phenotypic FQ micro-heteroresistance, the sensitivity remained high (91.2% (95% CI, 82.8%-99.6%)) and the specificity for subsequent FQ resistance was poor (7.1% (95% CI, 0.0%-21.3%) (Table 1). While FQ “pre-resistance” was detectable in multiple patient samples, significant heterogeneity of resistance loci occurred in the quinolone resistance-determining region (QRDR), frequently within the same patient. No samples with fixed RAVs (i.e., >95% subpopulation) for SLI were phenotypically susceptible, although multiple samples (n=5) with FQ RAV at 95% were reported as phenotypically susceptible, likely due to false-negative phenotypic DST calls, as noted elsewhere⁴⁵.

### CONCLUSIONS AND DISCUSSION

This study provides for an improved understanding of the dynamic processes occurring during the in vivo evolution of resistance and provides first-of-its-kind evidence that in vivo acquired resistance may be marked by a “pre-resistance” signal potentially detectable many months prior to onset of phenotypic resistance.

Through the use of high-resolution targeted sequencing (i.e., SMOR), we are able to detect early signals of impending resistance in select resistance loci for select drugs. While drug resistance phenotype testing and SMOR genotype analysis matched well for most samples, the presence of micro-heteroresistance caused diagnostic discordance, due to a number of “false positives.” However, importantly, we demonstrate that these genotype results, rather than being actual false positives, are actually a predictor of the treatment outcome (as measured by the final sample's phenotypic result), for at least aminoglycosides and fluoroquinolones. It is clear that below a certain threshold of a heteroresistant proportion, phenotypic tests will frequently miss the presence of resistant subpopulations.

In an early description of the impact of heteroresistance on sensitivity testing, Canetti et al. in 1963 defined a 1% or greater resistant sub-population of M. tuberculosis organisms on solid culture media as defining for clinical M. tb resistance⁴⁶. This threshold became codified in the proportion method, the most widely utilized phenotypic drug susceptibility test⁴⁷, as well as radiometric methods in liquid media (i.e., automated MGIT 960 systems). Yet, this interpretation is only loosely derived from clinical data, and physical characterization of such small proportions (i.e., single colonies) could not have been accurate (e.g., due to clumping). Recent investigations^{44,48,49} demonstrate that accurate MGIT phenotypic detection in the presence of M. tb heteroresistance often requires resistant sub-population proportions well-above the canonical 1% threshold, depending on the drug and specific RAV studied. In the present study utilizing ultra-deep sequencing, the optimal sub-population proportion threshold for concordance with phenotypic resistance for both aminoglycosides and fluoroquinolones was approximately 20%; above which, the genotype and phenotype matched 100% and below which a more stochastic effect may be present.

Without wishing to be bound by theory, our findings suggest that early genetic signals do occur in M.tb and are detectable many months before demonstrable phenotypic resistance as classically described. An investigation into whether augmentation of drug potency, concentration, and/or adherence at these early time points may mitigate subsequent treatment failure is needed.

Unless defined otherwise, all technical and scientific terms herein have the same meaning as commonly understood by one of ordinary skill in the art to which this invention belongs. All publications, patents, and patent publications cited are incorporated by reference herein in their entirety for all purposes.

It is understood that the disclosed invention is not limited to the particular methodology, protocols, and materials described as these can vary. It is also understood that the terminology used herein is for the purposes of describing particular embodiments only and is not intended to limit the scope of the present invention which will be limited only by the appended claims.

Those skilled in the art will recognize or be able to ascertain using no more than routine experimentation, many equivalents to the specific embodiments of the invention described herein. Such equivalents are intended to be encompassed by the following claims.

TABLE 1
Prognostic Value of SMOR-Determined
Micro-Heteroresistance for Final Resistant
Phenotype

| | |
|---|---|
| Sensitivity | Specificity |

rrs * (AUC .981; 95% CI .926-1.0)

| | | |
|---|---|---|
| 0.12% | 98.7% (96.1%, 100.0%) | 100% |
| 0.5% | 92.4% (84.9%, 99.9%) | 100% |
| 1% | 89.9% (81.6%, 98.1%) | 100% |
| 5% | 84.8% (74.3%, 95.3%) | 100% |
| 20% | 73.4% (56.6%, 90.2%) | 100% |

gyrA/B − high-level mutant** (AUC .761; 95% CI .563-.885)

| | | |
|---|---|---|
| 0.12% | 100% | 0% |
| 0.5% | 91.2% (82.8%, 99.6%) | 7.1% (0.0%, 21.3%) |
| 1% | 88.2% (77.7%, 98.7%) | 21.4% (1.9%, 40.9%) |
| 5% | 83.8% (74.1%, 93.6%) | 35.7% (20.3%, 51.1%) |
| 20% | 75.3% (60.2%, 86.9%) | 57.1% (15.0%, 99.3%) |

gyrA − low-level mutant** (AUC .724; 95% CI .423-.96)

| | | |
|---|---|---|
| 0.12% | 100% | 0% |
| 0.5% | 93.9% (86.4%, 100.0%) | 25.0% (0.0%, 71.4%) |
| 1% | 91.8% (83.8%, 99.9%) | 33.3% (0.0%, 71.1%) |
| 5% | 79.6% (64.4%, 94.8%) | 58.3% (7.0%, 100.0%) |
| 20% | 65.3% (38.2%, 92.4%) | 66.7% (25.6%, 100.0%) |

Observations with mutation percent <0.1% were not considered. ROC 95% CI were calculated via bias-corrected percentile bootstrap with resampling by the participant. Robust confidence intervals for the four performance measures could not be calculated if the numerator of the measure was zero, or the effective sample size for estimating it was one.
* rrs RAVs include 1401G, 1402T, 1484T.
**high-level RAVs include gyrA 94AAC, 94CAC, 94GGC, 94TAC; all gyrA88 mutations; and gyrB 496CTC, 500CAC; low-level RAVs include gyrA 90GTG, 91CCG, 94GC

TABLE 2
Non-limiting Examples of the Oligonucleotide Sequences Useful for Detecting
Single Nucleotide Polymorphisms (SNP) or Region of Interest (ROI) Associated with Drug Resistance

| | | SNP or | RDST or | | | SEQ ID |
|---|---|---|---|---|---|---|
| Drug | Gene | ROI | SMOR | Primer name | Oligonucleotide Sequence | NO: |
| Capreomycin | tlyA | both | SMOR | tlyA_1F_UT_CJA | ACCCAACTGAATGGAGCCACCGCC | 1 |
| | | | | | GTGTCCGAC | |
| Capreomycin | tlyA | both | SMOR | tlyA_1R_UT_CJA | ACGCACTTGACTTGTCTTCAGGACC | 2 |
| | | | | | ACCACCCGAGGAT | |
| Capreomycin | tlyA | both | SMOR | tlyA_2F_UT_CJA | ACCCAACTGAATGGAGCCGTTCATC | 3 |
| | | | | | TCGTTGGCTACCGTGTT | |
| Capreomycin | tlyA | both | SMOR | tlyA_2R_UT_CJA | ACGCACTTGACTTGTCTTCCCTTGA | 4 |
| | | | | | CGCCGACGCTGT | |
| Capreomycin | tlyA | both | SMOR | tlyA_3F_UT_CJA | ACCCAACTGAATGGAGCACAGCGT | 5 |
| | | | | | CGGCGTCAAGG | |
| Capreomycin | tlyA | both | SMOR | tlyA_3R_UT_CJA | ACGCACTTGACTTGTCTTCTGTGGA | 6 |
| | | | | | CGACCAGCAGAACACTG | |
| Streptomycin | gidB | ROI | SMOR | gidB_1F_UT_CJA | ACCCAACTGAATGGAGCGAGCCAG | 7 |
| | | | | | AACGCCGAGTC | |
| Streptomycin | gidB | ROI | SMOR | gidB_1R_UT_CJA | ACGCACTTGACTTGTCTTCGGTCCC | 8 |
| | | | | | ATAGCCTACCGACTTC | |
| Streptomycin | gidB | ROI | SMOR | gidB_2F_UT_CJA | ACCCAACTGAATGGAGCGAAGTCG | 9 |
| | | | | | GTAGGCTATGGGACC | |
| Streptomycin | gidB | ROI | SMOR | gidB_2R_UT_CJA | ACGCACTTGACTTGTCTTCCCCCGC | 10 |
| | | | | | ACGATCTCAACG | |
| Streptomycin | rpsL | ROI | SMOR | rpsLf-53 | ACCCAACTGAATGGAGCGGGGCAT | 11 |
| | | | | | GGCCGACAAACAGAACG | |
| Streptomycin | rpsL | ROI | SMOR | rpsLr210 | ACGCACTTGACTTGTCTTCCTCGCC | 12 |
| | | | | | GGGAATGTACGCCGTGAC | |
| Streptomycin | rpsL | ROI | SMOR | rpsLf150 | ACCCAACTGAATGGAGCAAGGTTG | 13 |
| | | | | | CCCGCGTGAAGTTGACGAGTC | |
| Streptomycin | rpsL | ROI | SMOR | rpsLr + 17 | ACGCACTTGACTTGTCTTCGGGCCC | 14 |
| | | | | | CTTGCGTGGCATCAGC | |
| Quinolones | gyrB | ROI | SMOR | gyrB_F_UT_CJA | ACCCAACTGAATGGAGCCGCAAGT | 15 |
| | | | | | CCGAACTGTATGTCGTAGAA | |
| Quinolones | gyrB | ROI | SMOR | gyrB_F2_UT_CJA | ACCCAACTGAATGGAGCGAGAGTT | 16 |
| | | | | | GGTGCGGCGTAAGAG | |
| Quinolones | gyrB | ROI | SMOR | gyrB_R_UT_CJA | ACGCACTTGACTTGTCTTCCCATCA | 17 |
| | | | | | GCACGATCTKGTGGTAGC | |
| Pyrazinamide | pncA | both | RDST | pncA_int1_F | ACCCAACTGAATGGAGCACGCTCCG | 18 |
| | | | | | GTGTAGGCAC | |
| Pyrazinamide | pncA | both | RDST | pncA_ext_R | ACGCACTTGACTTGTCTTCTATAGG | 19 |
| | | | | | GTCCATGACGCC | |
| Pyrazinamide | pncA | both | RDST | pncA_ext_F | ACCCAACTGAATGGAGCGTGAACA | 20 |
| | | | | | ACCCGACCCAG | |
| Pyrazinamide | pncA | both | RDST | pncA_int3_R | ACGCACTTGACTTGTCTTCGCCTCG | 21 |
| | | | | | ATTGCCGACGTGT | |
| Pyrazinamide | pncA | both | SMOR | pncAf-55 | ACCCAACTGAATGGAGCCTGCCGCG | 22 |
| | | | | | TCGGTAGGCAAACTGC | |
| Pyrazinamide | pncA | both | SMOR | pncAf102 | ACCCAACTGAATGGAGCCTGGCCG | 23 |
| | | | | | AAGCGGCGGACTACCATC | |
| Pyrazinamide | pncA | both | SMOR | pncAf223 | ACCCAACTGAATGGAGCGTACTCCC | 24 |
| | | | | | GGCGCGGACTTCCATCC | |
| Pyrazinamide | pncA | both | SMOR | pncAf361 | ACCCAACTGAATGGAGCGGCAACG | 25 |
| | | | | | CGGCGTCGATGAGGTC | |
| Pyrazinamide | pncA | both | SMOR | pncAr238 | ACGCACTTGACTTGTCTTCCCGCGC | 26 |
| | | | | | CGGGAGkACCGCTGAC | |
| Pyrazinamide | pncA | both | SMOR | pncAr344 | ACGCACTTGACTTGTCTTCGGCGTG | 27 |
| | | | | | CCGTTCTCGTCGACTCCTTC | |
| Pyrazinamide | pncA | both | SMOR | pncAr462 | ACGCACTTGACTTGTCTTCCCTGGT | 28 |
| | | | | | GGCCAAGCCATTGCGTACC | |
| Pyrazinamide | pncA | both | SMOR | pncAr + 44 | ACGCACTTGACTTGTCTTCGCGCTC | 29 |
| | | | | | CACCGCCGCCAACAG | |
| Ethambutol | embB | SNP | RDST | embB_F1 | ACCCAACTGAATGGAGCGCTGATTC | 30 |
| | | | | | CGGCAAGCTG | |
| Ethambutol | embB | SNP | RDST | embB_R1 | ACGCACTTGACTTGTCTTCGTGGAT | 31 |
| | | | | | GCGCCTGCCAGACC | |
| Ethambutol | embB | SNP | RDST | embB_F2 | ACCCAACTGAATGGAGCCCCATGTC | 32 |
| | | | | | AGCGACGCCAGTC | |
| Ethambutol | embB | SNP | RDST | embB_R2 | ACGCACTTGACTTGTCTTCCGCTGG | 33 |
| | | | | | TCACCTATGTGCTGATCG | |
| Isoniazid | ahpC | SNP | SMOR | ahpC-138f | ACCCAACTGAATGGAGCGGCCACG | 34 |
| | promoter | | | | GCCGGCTAGCACCTCTT | |
| Isoniazid | ahpC | SNP | SMOR | ahpC213r | ACGCACTTGACTTGTCTTCGAGCTT | 35 |
| | promoter | | | | GCTGAACGCCGCGATCTCG | |
| Isoniazid | ahpC | SNP | SMOR | ahpC-138f | ACCCAACTGAATGGAGCGGCCACG | 36 |
| | promoter | | | | GCCGGCTAGCACCTCTT | |
| Isoniazid | ahpC | SNP | SMOR | ahpC93r | ACGCACTTGACTTGTCTTCGGGCTG | 37 |
| | promoter | | | | CTTGGCGTCGACCTTGGA | |
| Isoniazid | ahpC | SNP | SMOR | ahpC-40f | ACCCAACTGAATGGAGCGCAACCA | 38 |
| | promoter | | | | AATGCATTGTCCGCTTTGATGAT | |
| Isoniazid | ahpC | SNP | SMOR | ahpC208r | ACGCACTTGACTTGTCTTCTGCTGA | 39 |
| | promoter | | | | ACGCCGCGATCTCGGTAGG | |
| Linezolid | rplC | SNP | SMOR | rplCf-44 | ACCCAACTGAATGGAGCGGCCAGC | 40 |
| | | | | | GTCGACGTCAACATCCAGTAG | |
| Linezolid | rplC | SNP | SMOR | rplCf118 | ACCCAACTGAATGGAGCGCACGCC | 41 |
| | | | | | CGAACGCGACGGTTAT | |
| Linezolid | rplC | SNP | SMOR | rplCf243 | ACCCAACTGAATGGAGCGCGGAGC | 42 |
| | | | | | TGCGGCTGGACGACTC | |
| Linezolid | rplC | SNP | SMOR | rplCf465 | ACCCAACTGAATGGAGCACGCCGG | 43 |
| | | | | | CGCGGGTGTTCA | |
| Linezolid | rplC | SNP | SMOR | rplCr259 | ACGCACTTGACTTGTCTTCCCAGCC | 44 |
| | | | | | GCAGCTCCGCCAGGTATC | |
| Linezolid | rplC | SNP | SMOR | rplCr322 | ACGCACTTGACTTGTCTTCCGGCGA | 45 |
| | | | | | AGATCTCCGCGGTCAACTCT | |
| Linezolid | rplC | SNP | SMOR | rplCr502 | ACGCACTTGACTTGTCTTCCGGCCA | 46 |
| | | | | | TCCGGGTGCCCTTGAA | |
| Linezolid | rplC | SNP | SMOR | rplCr + 49 | ACGCACTTGACTTGTCTTCGCCGGC | 47 |
| | | | | | GTCTTGACGTCGATTTTGAGT | |

SMOR: Single Molecule with Overlapping Reads Assay;
RDST: Rapid Drug Susceptibility Testing Assay

TABLE 3
Nucleotide Sequences of Genes Associated with Drug Resistance

| Gene Name | | SEQ ID |
|---|---|---|
| NCBI Ref. | Nucleotide Sequence | NO: |
| pncA | ATGCGGGCGTTGATCATCGTCGACGTGCAGAACGACTTCTGCGAGGGTGGCTCGCTGGCG | 48 |
| NC_000962.3 | GTAACCGGTGGCGCCGCGCTGGCCCGCGCCATCAGCGACTACCTGGCCGAAGCGGCGGAC | |
| | TACCATCACGTCGTGGCAACCAAGGACTTCCACATCGACCCGGGTGACCACTTCTCCGGC | |
| | ACACCGGACTATTCCTCGTCGTGGCCACCGCATTGCGTCAGCGGTACTCCCGGCGCGGACT | |
| | TCCATCCCAGTCTGGACACGTCGGCAATCGAGGCGGTGTTCTACAAGGGTGCCTACACCG | |
| | GAGCGTACAGCGGCTTCGAAGGAGTCGACGAGAACGGCACGCCACTGCTGAATTGGCTGC | |
| | GGCAACGCGGCGTCGATGAGGTCGATGTGGTCGGTATTGCCACCGATCATTGTGTGCGCC | |
| | AGACGGCCGAGGACGCGGTACGCAATGGCTTGGCCACCAGGGTGCTGGTGGACCTGACA | |
| | GCGGGTGTGTCGGCCGATACCACCGTCGCCGCGCTGGAGGAGATGCGCACCGCCAGCGTC | |
| | GAGTTGGTTTGCAGCTCCTGA | |
| tlyA | GTGGCACGACGTGCCCGCGTTGACGCCGAGCTAGTCCGGCGGGGCCTGGCGCGATCACGT | 49 |
| NC_000962.3 | CAACAGGCCGCGGAGTTGATCGGCGCCGGCAAGGTGCGCATCGACGGGCTGCCGGCGGT | |
| | CAAGCCGGCCACCGCCGTGTCCGACACCACCGCGCTGACCGTGGTGACCGACAGTGAACG | |
| | CGCCTGGGTATCGCGCGGAGCGCACAAACTAGTCGGTGCGCTGGAGGCGTTCGCGATCGC | |
| | GGTGGCGGGCCGGCGCTGTCTGGACGCGGGCGCATCGACCGGTGGGTTCACCGAAGTACT | |
| | GCTGGACCGTGGTGCCGCCCACGTGGTGGCCGCCGATGTCGGATACGGCCAGCTGGCGTG | |
| | GTCGCTGCGCAACGATCCTCGGGTGGTGGTCCTCGAGCGGACCAACGCACGTGGCCTCAC | |
| | ACCGGAGGCGATCGGCGGTCGCGTCGACCTGGTAGTGGCCGACCTGTCGTTCATCTCGTT | |
| | GGCTACCGTGTTGCCCGCGCTGGTTGGATGCGCTTCGCGCGACGCCGATATCGTTCCACTG | |
| | GTGAAGCCGCAGTTTGAGGTGGGGAAAGGTCAGGTCGGCCCCGGTGGGGTGGTCCATGAC | |
| | CCGCAGTTGCGTGCGCGGTCGGTGCTCGCGGTCGCGCGGCGGGCACAGGAGCTGGGCTGG | |
| | CACAGCGTCGGCGTCAAGGCCAGCCCGCTGCCGGGCCCATCGGGCAATGTCGAGTACTTC | |
| | CTGTGGTTGCGCACGCAGACCGACCGGGCATTGTCGGCCAAGGGATTGGAGGATGCGGTG | |
| | CACCGTGCGATTAGCGAGGGCCCGTAG | |
| gidB | TCACGCCGTCCCTCCACTCGCCATCCGTGCCGACCCTCGGGCGATCTGCTTTCCACGTCGT | 50 |
| NC_000962.3 | GCGAACACCACGGTCGCGGGCGGACGCAAATAGTTCGCGCCACATGTCACCACCCTGACA | |
| | TCAACCGCGCCCGATGCGATCATCACACGCCGGTGCTCCCGTACTTCGTCGTGAGCCCGCT | |
| | CGCCTTTGATGGCGAGCATTCGCCCGTTCGGCCGTATCAACGGCATGCTCCATTTCGTCAA | |
| | CTTGTCCAACGCGGCCACCGCCCGTGACACCGCAGCGTCGCTGCCGCCCAATTGGTCCTGC | |
| | ACCCAGGACTCCTCGGCGCGCCCCCGCACGATCTCAACGGCCACGCCCAGATCTGTCACC | |
| | ATCTCTCGAAGAAACTCGGTGCGGCGCAGTAGCGGTTCTAGGAGAACTACCTGGAGGTCC | |
| | GGCCGCGCTATCGCCAATGGCACGCCCGGCAACCCGGCTCCGCTACCGATATCCACGACC | |
| | CGGTCACCGCGTTCGAGGAGCTCACCGATCACGGCGCAGTTCAGTAGATGCCGGTCCCAT | |
| | AGCCTACCGACTTCGCGGGGTCCCACCAGCCCCCGCTCCACACCGGGTCCCGCCAACGCT | |
| | TCGGCGTACCGCCGAGCAAGGCCAAGCCGCGGTCCGAAGATCGCAGACGCCGCGGGCTC | |
| | GATCGGAGACAT | |
| rpsL | ATGCCAACCATCCAGCAGCTGGTCCGCAAGGGTCGTCGGGACAAGATCAGTAAGGTCAAG | 51 |
| NC_000962.3 | ACCGCGGCTCTGAAGGGCAGCCCGCAGCGTCGTGGTGTATGCACCCGCGTGTACACCACC | |
| | ACTCCGAAGAAGCCGAACTCGGCGCTTCGGAAGGTTGCCCGCGTGAAGTTGACGAGTCAG | |
| | GTCGAGGTCACGGCGTACATTCCCGGCGAGGGCCACAACCTGCAGGAGCACTCGATGGTG | |
| | CTGGTGCGCGGCGGCCGGGTGAAGGACCTGCCTGGTGTGCGCTACAAGATCATCCGCGGT | |
| | TCGCTGGATACGCAGGGTGTCAAGAACCGCAAACAGGCACGCAGCCGTTACGGCGCTAA | |
| | GAAGGAGAAGGGCTGA | |
| gyrB | GTGGCTGCCCAGAAAAAGAAGGCCCAAGACGAATACGGCGCTGCGTCTATCACCATTCTC | 52 |
| NC_000962.3 | GAAGGGCTGGAGGCCGTCCGCAAACGTCCCGGCATGTACATTGGCTCGACCGGTGAGCGC | |
| | GGTTTACACCATCTCATTTGGGAGGTGGTCGACAACGCGGTCGACGAGGCGATGGCCGGT | |
| | TATGCAACCACAGTGAACGTAGTGCTGCTTGAGGATGGCGGTGTCGAGGTCGCCGACGAC | |
| | GGCCGCGGCATTCCGGTCGCCACCCACGCCTCCGGCATACCGACCGTCGACGTGGTGATG | |
| | ACACAACTACATGCCGGCGGCAAGTTCGACTCGGACGCGTATGCGATATCTGGTGGTCTG | |
| | CACGGCGTCGGCGTGTCGGTGGTTAACGCGCTATCCACCCGGCTCGAAGTCGAGATCAAG | |
| | CGCGACGGGTACGAGTGGTCTCAGGTTTATGAGAAGTCGGAACCCCTGGGCCTCAAGCAA | |
| | GGGGCGCCGACCAAGAAGACGGGGTCAACGGTGCGGTTCTGGGCCGACCCCGCTGTTTTC | |
| | GAAACCACGGAATACGACTTCGAAACCGTCGCCCGCCGGCTGCAAGAGATGGCGTTCCTC | |
| | AACAAGGGGCTGACCATCAACCTGACCGACGAGAGGGTGACCCAAGACGAGGTCGTCGA | |
| | CGAAGTGGTCAGCGACGTCGCCGAGGCGCCGAAGTCGGCAAGTGAACGCGCAGCCGAAT | |
| | CCACTGCACCGCACAAAGTTAAGAGCCGCACCTTTCACTATCCGGGTGGCCTGGTGGACT | |
| | TCGTGAAACACATCAACCGCACCAAGAACGCGATTCATAGCAGCATCGTGGACTTTTCCG | |
| | GCAAGGGCACCGGGCACGAGGTGGAGATCGCGATGCAATGGAACGCCGGGTATTCGGAG | |
| | TCGGTGCACACCTTCGCCAACACCATCAACACCCACGAGGGCGGCACCCACGAAGAGGGC | |
| | TTCCGCAGCGCGCTGACGTCGGTGGTGAACAAGTACGCCAAGGACCGCAAGCTACTGAAG | |
| | GACAAGGACCCCAACCTCACCGGTGACGATATCCGGGAAGGCCTGGCCGCTGTGATCTCG | |
| | GTGAAGGTCAGCGAACCGCAGTTCGAGGGCCAGACCAAGACCAAGTTGGGCAACACCGA | |
| | GGTCAAATCGTTTGTGCAGAAGGTCTGTAACGAACAGCTGACCCACTGGTTTGAAGCCAA | |
| | CCCCACCGACGCGAAAGTCGTTGTGAACAAGGCTGTGTCCTCGGCGCAAGCCCGTATCGC | |
| | GGCACGTAAGGCACGAGAGTTGGTGCGGCGTAAGAGCGCCACCGACATCGGTGGATTGC | |
| | CCGGCAAGCTGGCCGATTGCCGTTCCACGGATCCGCGCAAGTCCGAACTGTATGTCGTAG | |
| | AAGGTGACTCGGCCGGCGGTTCTGCAAAAAGCGGTCGCGATTCGATGTTCCAGGCGATAC | |
| | TTCCGCTGCGCGGCAAGATCATCAATGTGGAGAAAGCGCGCATCGACCGGGTGCTAAAGA | |
| | ACACCGAAGTTCAGGCGATCATCACGGCGCTGGGCACCGGGATCCACGACGAGTTCGATA | |
| | TCGGCAAGCTGCGCTACCACAAGATCGTGCTGATGGCCGACGCCGATGTTGACGGCCAAC | |
| | ATATTTCCACGCTGTTGTTGACGTTGTTGTTCCGGTTCATGCGGCCGCTCATCGAGAACGG | |
| | GCATGTGTTTTTGGCACAACCGCCGCTGTACAAACTCAAGTGGCAGCGCAGTGACCCGGA | |
| | ATTCGCATACTCCGACCGCGAGCGCGACGGTCTGCTGGAGGCGGGGCTGAAGGCCGGGA | |
| | AGAAGATCAACAAGGAAGACGGCATTCAGCGGTACAAGGGTCTAGGTGAAATGGACGCT | |
| | AAGGAGTTGTGGGAGACCACCATGGATCCCTCGGTTCGTGTGTTGCGTCAAGTGACGCTG | |
| | GACGACGCCGCCGCCGCCGACGAGTTGTTCTCCATCCTGATGGGCGAGGACGTCGACGCG | |
| | CGGCGCAGCTTTATCACCCGCAACGCCAAGGATGTTCGGTTCCTGGATGTCTAA | |
| embB | ATGACACAGTGCGCGAGCAGACGCAAAAGCACCCCAAATCGGGCGATTTTGGGGGCTTTT | 53 |
| NC_000962.3 | GCGTCTGCTCGCGGGACGCGCTGGGTGGCCACCATCGCCGGGCTGATTGGCTTTGTGTTGT | |
| | CGGTGGCGACGCCGCTGCTGCCCGTCGTGCAGACCACCGCGATGCTCGACTGGCCACAGC | |
| | GGGGGCAACTGGGCAGCGTGACCGCCCCGCTGATCTCGCTGACGCCGGTCGACTTTACCG | |
| | CCACCGTGCCGTGCGACGTGGTGCGCGCCATGCCACCCGCGGGCGGGGTGGTGCTGGGCA | |
| | CCGCACCCAAGCAAGGCAAGGACGCCAATTTGCAGGCGTTGTTCGTCGTCGTCAGCGCCC | |
| | AGCGCGTGGACGTCACCGACCGCAACGTGGTGATCTTGTCCGTGCCGCGCGAGCAGGTGA | |
| | CGTCCCCGCAGTGTCAACGCATCGAGGTCACCTCTACCCACGCCGGCACCTTCGCCAACTT | |
| | CGTCGGGCTCAAGGACCCGTCGGGCGCGCCGCTGCGCAGCGGCTTCCCCGACCCCAACCT | |
| | GCGCCCGCAGATTGTCGGGGTGTTCACCGACCTGACCGGGCCCGCGCCGCCCGGGCTGGC | |
| | GGTCTCGGCGACCATCGACACCCGGTTCTCCACCCGGCCGACCACGCTGAAACTGCTGGC | |
| | GATCATCGGGGCGATCGTGGCCACCGTCGTCGCACTGATCGCGTTGTGGCGCCTGGACCA | |
| | GTTGGACGGGCGGGGCTCAATTGCCCAGCTCCTCCTCAGGCCGTTCCGGCCTGCATCGTCG | |
| | CCGGGCGGCATGCGCCGGCTGATTCCGGCAAGCTGGCGCACCTTCACCCTGACCGACGCC | |
| | GTGGTGATATTCGGCTTCCTGCTCTGGCATGTCATCGGCGCGAATTCGTCGGACGACGGCT | |
| | ACATCCTGGGCATGGCCCGAGTCGCCGACCACGCCGGCTACATGTCCAACTATTTCCGCTG | |
| | GTTCGGCAGCCCGGAGGATCCCTTCGGCTGGTATTACAACCTGCTGGCGCTGATGACCCAT | |
| | GTCAGCGACGCCAGTCTGTGGATGCGCCTGCCAGACCTGGCCGCCGGGCTAGTGTGCTGG | |
| | CTGCTGCTGTCGCGTGAGGTGCTGCCCCGCCTCGGGCCGGCGGTGGAGGCCAGCAAACCC | |
| | GCCTACTGGGCGGCGGCCATGGTCTTGCTGACCGCGTGGATGCCGTTCAACAACGGCCTG | |
| | CGGCCGGAGGGCATCATCGCGCTCGGCTCGCTGGTCACCTATGTGCTGATCGAGCGGTCC | |
| | ATGCGGTACAGCCGGCTCACACCGGCGGCGCTGGCCGTCGTTACCGCCGCATTCACACTG | |
| | GGTGTGCAGCCCACCGGCCTGATCGCGGTGGCCGCGCTGGTGGCCGGCGGCCGCCCGATG | |
| | CTGCGGATCTTGGTGCGCCGTCATCGCCTGGTCGGCACGTTGCCGTTGGTGTCGCCGATGC | |
| | TGGCCGCCGGCACCGTCATCCTGACCGTGGTGTTCGCCGACCAGACCCTGTCAACGGTGTT | |
| | GGAAGCCACCAGGGTTCGCGCCAAAATCGGGCCGAGCCAGGCGTGGTATACCGAGAACC | |
| | TGCGTTACTACTACCTCATCCTGCCCACCGTCGACGGTTCGCTGTCGCGGCGCTTCGGCTT | |
| | TTTGATCACCGCGCTATGCCTGTTCACCGCGGTGTTCATCATGTTGCGGCGCAAGCGAATT | |
| | CCCAGCGTGGCCCGCGGACCGGCGTGGCGGCTGATGGGCGTCATCTTCGGCACCATGTTC | |
| | TTCCTGATGTTCACGCCCACCAAGTGGGTGCACCACTTCGGGCTGTTCGCCGCCGTAGGGG | |
| | CGGCGATGGCCGCGCTGACGACGGTGTTGGTATCCCCATCGGTGCTGCGCTGGTCGCGCA | |
| | ACCGGATGGCGTTCCTGGCGGCGTTATTCTTCCTGCTGGCGTTGTGTTGGGCCACCACCAA | |
| | CGGCTGGTGGTATGTCTCCAGCTACGGTGTGCCGTTCAACAGCGCGATGCCGAAGATCGA | |
| | CGGGATCACAGTCAGCACAATCTTTTTCGCCCTGTTTGCGATCGCCGCCGGCTATGCGGCC | |
| | TGGCTGCACTTCGCGCCCCGCGGCGCCGGCGAAGGGCGGCTGATCCGCGCGCTGACGACA | |
| | GCCCCGGTACCGATCGTGGCCGGTTTCATGGCGGCGGTGTTCGTCGCGTCCATGGTGGCCG | |
| | GGATCGTGCGACAGTACCCGACCTACTCCAACGGCTGGTCCAACGTGCGGGCGTTTGTCG | |
| | GCGGCTGCGGACTGGCCGACGACGTACTCGTCGAGCCTGATACCAATGCGGGTTTCATGA | |
| | AGCCGCTGGACGGCGATTCGGGTTCTTGGGGCCCCTTGGGCCCGCTGGGTGGAGTCAACC | |
| | CGGTCGGCTTCACGCCCAACGGCGTACCGGAACACACGGTGGCCGAGGCGATCGTGATGA | |
| | AACCCAACCAGCCCGGCACCGACTACGACTGGGATGCGCCGACCAAGCTGACGAGTCCTG | |
| | GCATCAATGGTTCTACGGTGCCGCTGCCCTATGGGCTCGATCCCGCCCGGGTACCGTTGGC | |
| | AGGCACCTACACCACCGGCGCACAGCAACAGAGCACACTCGTCTCGGCGTGGTATCTCCT | |
| | GCCTAAGCCGGACGACGGGCATCCGCTGGTCGTGGTGACCGCCGCGGGCAAGATCGCCGG | |
| | CAACAGCGTGCTGCACGGGTACACCCCCGGGCAGACTGTGGTGCTCGAATACGCCATGCC | |
| | GGGACCCGGAGCGCTGGTACCCGCCGGGCGGATGGTGCCCGACGACCTATACGGAGAGC | |
| | AGCCCAAGGCGTGGCGCAACCTGCGCTTCGCCCGAGCAAAGATGCCCGCCGATGCCGTCG | |
| | CGGTCCGGGTGGTGGCCGAGGATCTGTCGCTGACACCGGAGGACTGGATCGCGGTGACCC | |
| | CGCCGCGGGTACCGGACCTGCGCTCACTGCAGGAATATGTGGGCTCGACGCAGCCGGTGC | |
| | TGCTGGACTGGGCGGTCGGTTTGGCCTTCCCGTGCCAGCAGCCGATGCTGCACGCCAATG | |
| | GCATCGCCGAAATCCCGAAGTTCCGCATCACACCGGACTACTCGGCTAAGAAGCTGGACA | |
| | CCGACACGTGGGAAGACGGCACTAACGGCGGCCTGCTCGGGATCACCGACCTGTTGCTGC | |
| | GGGCCCACGTCATGGCCACCTACCTGTCCCGCGACTGGGCCCGCGATTGGGGTTCCCTGC | |
| | GCAAGTTCGACACCCTGGTCGATGCCCCTCCCGCCCAGCTCGAGTTGGGCACCGCGACCC | |
| | GCAGCGGCCTGTGGTCACCGGGCAAGATCCGAATTGGTCCATAG | |
| rplC | ATGGCACGAAAGGGCATTCTCGGTACCAAGCTGGGTATGACGCAGGTATTCGACGAAAGC | 54 |
| NC_000962.3 | AACAGAGTAGTACCGGTGACCGTGGTCAAGGCCGGGCCCAACGTGGTAACCCGCATCCGC | |
| | ACGCCCGAACGCGACGGTTATAGCGCCGTGCAGCTGGCCTATGGCGAGATCAGCCCACGC | |
| | AAGGTCAACAAGCCGCTGACAGGTCAGTACACCGCCGCCGGCGTCAACCCACGCCGATAC | |
| | CTGGCGGAGCTGCGGCTGGACGACTCGGATGCCGCGACCGAGTACCAGGTTGGGCAAGA | |
| | GTTGACCGCGGAGATCTTCGCCGATGGCAGCTACGTCGATGTGACGGGTACCTCCAAGGG | |
| | CAAAGGTTTCGCCGGCACCATGAAGCGGCACGGCTTCCGCGGTCAGGGCGCCAGTCACGG | |
| | TGCCCAGGCGGTGCACCGCCGTCCGGGCTCCATCGGCGGATGTGCCACGCCGGCGCGGGT | |
| | GTTCAAGGGCACCCGGATGGCCGGGCGGATGGGCAATGACCGGGTGACCGTTCTTAACCT | |
| | TTTGGTGCATAAGGTCGATGCCGAGAACGGCGTGCTGCTGATCAAGGGTGCGGTTCCTGG | |
| | CCGCACCGGTGGACTGGTCATGGTCCGCAGTGCGATCAAACGAGGTGAGAAGTGA | |

TABLE 4
Protein Sequences of Genes Associated with Drug Resistance

| Gene Name | | SEQ ID |
|---|---|---|
| NCBI Ref. | Amino Acid Sequence | NO: |
| pncA | MRALIIVDVQNDFCEGGSLAVTGGAALARAISDYLAEAADYHHVVATKDFHIDPGDHFSGTP | 55 |
| NP_216559.1 | DYSSSWPPHCVSGTPGADFHPSLDTSALEAVFYKGAYTGAYSGFEGVDENGTPLLNWLRQRG | |
| | VDEVDVVGIATDHCVRQTAEDAVRNGLATRVLVDLTAGVSADTTVAALEEMRTASVELVCS | |
| | S | |
| tlyA | MARRARVDAELVRRGLARSRQQAAELIGAGKVRIDGLPAVKPATAVSDTTALTVVTDSERA | 56 |
| NP_216210.1 | WVSRGAHKLVGALEAFAIAVAGRRCLDAGASTGGFTEVLLDRGAAHVVAADVGYGQLAW | |
| | SLRNDPRVVVLERTNARGLTPEAIGGRVDLVVADLSFISLATVLPALVGCASRDADIVPLVKP | |
| | QFEVGKGQVGPGGVVHDPQLRARSVLAVARRAQELGWHSVGVKASPLPGPSGNVEYFLWL | |
| | RTQTDRALSAKGLEDAVHRAISEGP | |
| gidB | MSPIEPAASAIFGPRLGLARRYAEALAGPGVERGLVGPREVGRLWDRHLLNCAVIGELLERG | 57 |
| NP_218436.2 | DRVVDIGSGAGLPGVPLAIARPDLQVVLLEPLLRRTEFLREMVTDLGVAVEIVRGRAEESWV | |
| | QDQLGGSDAAVSRAVAALDKLTKWSMPLIRPNGRMLAIKGERAHDEVREHRRVMIASGAV | |
| | DVRVVTCGANYLRPPATVVFARRGKQIARGSARMASGGTA | |
| rpsL | MPTIQQLVRKGRRDKISKVKTAALKGSPQRRGVCTRVYTTTPKKPNSALRKVARVKLTSQVE | 58 |
| NP_215196.1 | VTAYIPGEGHNLQEHSMVLVRGGRVKDLPGVRYKIIRGSLDTQGVKNRKQARSRYGAKKEK | |
| | G | |
| gyrB | MAAQKKKAQDEYGAASITILEGLEAVRKRPGMYIGSTGERGLHHLIWEVVDNAVDEAMAG | 59 |
| NP_214519.2 | YATTVNVVLLEDGGVEVADDGRGIPVATHASGIPTVDVVMTQLHAGGKFDSDAYAISGGLH | |
| | GVGVSVVNALSTRLEVEIKRDGYEWSQVYEKSEPLGLKQGAPTKKTGSTVRFWADPAVFET | |
| | TEYDFETVARRLQEMAFLNKGLTINLTDERVTQDEVVDEVVSDVAEAPKSASERAAESTAPH | |
| | KVKSRTFHYPGGLVDFVKHINRTKNAIHSSIVDFSGKGTGHEVEIAMQWNAGYSESVHTFAN | |
| | TINTHEGGTHEEGFRSALTSVVNKYAKDRKLLKDKDPNLTGDDIREGLAAVISVKVSEPQFEG | |
| | QTKTKLGNTEVKSFVQKVCNEQLTHWFEANPTDAKVVVNKAVSSAQARIAARKARELVRR | |
| | KSATDIGGLPGKLADCRSTDPRKSELYVVEGDSAGGSAKSGRDSMFQAILPLRGKIINVEKAR | |
| | IDRVLKNTEVQAIITALGTGIHDEFDIGKLRYHKIVLMADADVDGQHISTLLLTLLFRFMRPLIE | |
| | NGHVFLAQPPLYKLKWQRSDPEFAYSDRERDGLLEAGLKAGKKINKEDGIQRYKGLGEMDA | |
| | KELWETTMDPSVRVLRQVTLDDAAAADELFSILMGEDVDARRSFITRNAKDVRFLDV | |
| embB | MTQCASRRKSTPNRAILGAFASARGTRWVATIAGLIGFVLSVATPLLPVVQTTAMLDWPQRG | 60 |
| NP_218312.1 | QLGSVTAPLISLTPVDFTATVPCDVVRAMPPAGGVVLGTAPKQGKDANLQALFVVVSAQRV | |
| | DVTDRNVVILSVPREQVTSPQCQRIEVTSTHAGTFANFVGLKDPSGAPLRSGFPDPNLRPQIVG | |
| | VFTDLTGPAPPGLAVSATIDTRFSTRPTTLKLLAIIGAIVATVVALIALWRLDQLDGRGSIAQLL | |
| | LRPFRPASSPGGMRRLIPASWRTFTLTDAVVIFGFLLWHVIGANSSDDGYILGMARVADHAG | |
| | YMSNYFRWFGSPEDPFGWYYNLLALMTHVSDASLWMRLPDLAAGLVCWLLLSREVLPRLG | |
| | PAVEASKPAYWAAAMVLLTAWMPFNNGLRPEGIIALGSLVTYVLIERSMRYSRLTPAALAVV | |
| | TAAFTLGVQPTGLIAVAALVAGGRPMLRILVRRHRLVGTLPLVSPMLAAGTVILTVVFADQT | |
| | LSTVLEATRVRAKIGPSQAWYTENLRYYYLILPTVDGSLSRRFGFLITALCLFTAVFIMLRRKR | |
| | IPSVARGPAWRLMGVIFGTMFFLMFTPTKWVHHFGLFAAVGAAMAALTTVLVSPSVLRWSR | |
| | NRMAFLAALFFLLALCWATTNGWWYVSSYGVPFNSAMPKIDGITVSTIFFALFAIAAGYAAW | |
| | LHFAPRGAGEGRLIRALTTAPVPIVAGFMAAVFVASMVAGIVRQYPTYSNGWSNVRAFVGG | |
| | CGLADDVLVEPDTNAGFMKPLDGDSGSWGPLGPLGGVNPVGFTPNGVPEHTVAEAIVMKPN | |
| | QPGTDYDWDAPTKLTSPGINGSTVPLPYGLDPARVPLAGTYTTGAQQQSTLVSAWYLLPKPD | |
| | DGHPLVVVTAAGKIAGNSVLHGYTPGQTVVLEYAMPGPGALVPAGRMVPDDLYGEQPKAW | |
| | RNLRFARAKNIPADAVAVRVVAEDLSLTPEDWIAVTPPRVPDLRSLQEYVGSTQPVLLDWAV | |
| | GLAFPCQQPMLHANGIAEIPKFRITPDYSAKKLDTDTWEDGTNGGLLGITDLLLRAHVMATY | |
| | LSRDWARDWGSLRKFDTLVDAPPAQLELGTATRSGLWSPGKIRIGP | |
| rplC | MARKGILGTKLGMTQVFDESNRVVPVTVVKAGPNVVTRIRTPERDGYSAVQLAYGEISPRKV | 61 |
| NP_215215.1 | NKPLTGQYTAAGVNPRRYLAELRLDDSDAATEYQVGQELTAEIFADGSYVDVTGTSKGKGF | |
| | AGTMKRHGFRGQGASHGAQAVHRRPGSIGGCATPARVFKGTRMAGRIVIGNDRVTVLNLLV | |
| | HKVDAENGVLLIKGAVPGRTGGLVMVRSAIKRGEK | |

### REFERENCES

1. Warren R, de Kock M, Engelke E, et al. Safe Mycobacterium tuberculosis DNA extraction method that does not compromise integrity. Journal of clinical microbiology 2006; 44:254-56.
2. Black P A, de Vos M, Louw G E, et al. Whole genome sequencing reveals genomic heterogeneity and antibiotic purification in Mycobacterium tuberculosis isolates. BMC genomics 2015; 16:857.
3. Milne I, Bayer M, Cardle L, et al. Tablet—next generation sequence assembly visualization.

Bioinformatics 2010; 26:401-2.
4. Coll F, McNerney R, Preston M D, et al. Rapid determination of anti-tuberculosis drug resistance from whole-genome sequences. Genome Med 2015; 7:51.
5. Colman R E, Anderson J, Lemmer D, et al. Rapid Drug Susceptibility Testing of Drug-Resistant Mycobacterium tuberculosis Isolates Directly from Clinical Samples by Use of Amplicon Sequencing: a Proof-of-Concept Study. Journal of clinical microbiology 2016; 54:2058-67.
6. Langmead B. Aligning short sequencing reads with Bowtie. Curr Protoc Bioinformatics 2010; Chapter 11:Unit 11 7.
7. Li H, Durbin R. Fast and accurate short read alignment with Burrows-Wheeler transform. Bioinformatics 2009; 25:1754-60.
8. Springer B, Lucke K, Calligaris-Maibach R, Ritter C, Bottger E C. Quantitative drug susceptibility testing of Mycobacterium tuberculosis by use of MGIT 960 and EpiCenter instrumentation. Journal of clinical microbiology 2009; 47:1773-80.
9. Schnippel K, Ndjeka N, Maartens G, et al. Effect of bedaquiline on mortality in South African patients with drug-resistant tuberculosis: a retrospective cohort study. The Lancet Respiratory medicine 2018; 6:699-706.
10. Ferlazzo G, Mohr E, Laxmeshwar C, et al. Early safety and efficacy of the combination of bedaquiline and delamanid for the treatment of patients with drug-resistant tuberculosis in Armenia, India, and South Africa: a retrospective cohort study. The Lancet Infectious diseases 2018; 18:536-44.
11. Conradie F, Meintjes G, Hughes J, et al. Clinical access to Bedaquiline Programme for the treatment of drug-resistant tuberculosis. S Afr Med J 2014; 104:164-66.
12. Andries K, Villellas C, Coeck N, et al. Acquired resistance of Mycobacterium tuberculosis to bedaquiline. PloS one 2014; 9:e102135.
13. McLeay S C, Vis P, van Heeswijk R P, Green B. Population pharmacokinetics of bedaquiline (TMC207), a novel antituberculosis drug. Antimicrobial agents and chemotherapy 2014; 58:5315-24.
14. Meftahi N, Namouchi A, Mhenni B, Brandis G, Hughes D, Mardassi H. Evidence for the critical role of a secondary site rpoB mutation in the compensatory evolution and successful transmission of an MDR tuberculosis outbreak strain. J Antimicrob Chemother 2016; 71:324-32.
15. Comas I, Borrell S, Roetzer A, et al. Whole-genome sequencing of rifampicin-resistant Mycobacterium tuberculosis strains identifies compensatory mutations in RNA polymerase genes. Nature Genetics 2012; 44:106-10.
16. de Vos M, Muller B, Borrell S, et al. Putative compensatory mutations in the rpoC gene of rifampin-resistant Mycobacterium tuberculosis are associated with ongoing transmission. Antimicrobial agents and chemotherapy 2013; 57:827-32.
17. Brandis G, Hughes D. Genetic characterization of compensatory evolution in strains carrying rpoB Ser531Leu, the rifampicin resistance mutation most frequently found in clinical isolates. J Antimicrob Chemother 2013; 68:2493-97.
18. Brandis G, Wrande M, Liljas L, Hughes D. Fitness-compensatory mutations in rifampicin-resistant RNA polymerase. Mol Microbiol 2012; 85:142-51.
19. Cegielski J P, Kurbatova E, van der Walt M, et al. Multidrug-Resistant Tuberculosis Treatment Outcomes in Relation to Treatment and Initial Versus Acquired Second-Line Drug Resistance. Clin Infect Dis. 2016; 62(4):418-430.
20. Angeby K, Jureen P, Kahlmeter G, Hoffner S E, Schon T. Challenging a dogma: antimicrobial susceptibility testing breakpoints for Mycobacterium tuberculosis. Bull World Health Organ. 2012; 90(9):693-698.
21. Folkvardsen D B, Svensson E, Thomsen V O, et al. Can molecular methods detect 1% isoniazid resistance in Mycobacterium tuberculosis? J Clin Microbiol. 2013; 51(5):1596-1599.
22. Chakravorty S, Simmons A M, Rowneki M, Parmar H, Cao Y, Ryan J, Banada P P, Deshpande S, Shenai S, Gall A, Glass J, Krieswirth B, Schumacher S G, Nabeta P, Tukvadze N, Rodrigues C, Skrahina A, Tagliani E, Cirillo D M, Davidow A, Denkinger C M, Persing D, Kwiatkowski R, Jones M, Alland D. 2017. The new Xpert MTB/RIF Ultra: improving detection of Mycobacterium tuberculosis and resistance to rifampin in an assay suitable for point-of-care testing. mBio 8:e00812-17. https://doi.org/10.1128/mBio.00812-17.
23. Blakemore R, Story E, Helb D, et al. Evaluation of the analytical performance of the Xpert MTB/RIF assay. J Clin Microbiol. 2010; 48(7):2495-2501.
24. Colman R E, Schupp J M, Hicks N D, et al. Detection of Low-Level Mixed-Population Drug Resistance in Mycobacterium tuberculosis Using High Fidelity Amplicon Sequencing. PLoS One. 2015; 10(5):e0126626.
25. Dheda K, Gumbo T, Maartens G, et al. The epidemiology, pathogenesis, transmission, diagnosis, and management of multidrug-resistant, extensively drug-resistant, and incurable tuberculosis. Lancet Respir Med. 2017.
26. Kyeyune F, Gibson R M, Nankya I, et al. Low-Frequency Drug Resistance in HIV-Infected Ugandans on Antiretroviral Treatment Is Associated with Regimen Failure. Antimicrob Agents Chemother. 2016; 60(6):3380-3397.
27. Li J Z, Kuritzkes D R. Clinical implications of HIV-1 minority variants. Clin Infect Dis. 2013; 56(11):1667-1674.
28. Wang J, Cazzato E, Ladewig E, et al. Clonal evolution of glioblastoma under therapy. Nat Genet. 2016; 48(7):768-776.
29. Zhang J, Fujimoto J, Zhang J, et al. Intratumor heterogeneity in localized lung adenocarcinomas delineated by multiregion sequencing. Science. 2014; 346(6206):256-259.
30. Rinder H. Hetero-resistance: an under-recognised confounder in diagnosis and therapy? J Med Microbiol. 2001; 50(12):1018-1020.
31. Ford C B, Lin P L, Chase M R, et al. Use of whole genome sequencing to estimate the mutation rate of Mycobacterium tuberculosis during latent infection. Nat Genet. 2011; 43(5):482-486.
32. Rinder H, Mieskes K T, Loscher T. Heteroresistance in Mycobacterium tuberculosis. Int J Tuberc Lung Dis. 2001; 5(4):339-345.
33. Chakravorty S, Kothari H, Aladegbami B, et al. Rapid, high-throughput detection of rifampin resistance and heteroresistance in Mycobacterium tuberculosis by use of sloppy molecular beacon melting temperature coding. J Clin Microbiol. 2012; 50(7):2194-2202.
34. Nikolayevskyy V, Balabanova Y, Simak T, Malomanova N, Fedorin I, Drobniewski F. Performance of the Genotype MTBDRPlus assay in the diagnosis of tuberculosis and drug resistance in Samara, Russian Federation. BMC Clin Pathol. 2009; 9:2.
35. Streicher E M, Bergval I, Dheda K, et al. Mycobacterium tuberculosis population structure determines the outcome of genetics-based second-line drug resistance testing. Antimicrob Agents Chemother. 2012; 56(5):2420-2427.
36. Zhang X, Zhao B, Liu L, Zhu Y, Zhao Y, Jin Q. Subpopulation analysis of heteroresistance to fluoroquinolone in Mycobacterium tuberculosis isolates from Beijing, China. J Clin Microbiol. 2012; 50(4):1471-1474.
37. Zhang X, Zhao B, Huang H, et al. Co-occurrence of amikacin-resistant and -susceptible Mycobacterium tuberculosis isolates in clinical samples from Beijing, China. J Antimicrob Chemother. 2013; 68(7):1537-1542.
38. Georghiou S B, Seifert M, Catanzaro D G, Garfein R S, Rodwell T C. Increased Tuberculosis Patient Mortality Associated with Mycobacterium tuberculosis Mutations Conferring Resistance to Second-Line Antituberculous Drugs. J Clin Microbiol. 2017; 55(6):1928-1937.
39. Koser C U, Bryant J M, Becq J, et al. Whole-genome sequencing for rapid susceptibility testing of M. tuberculosis. N Engl J Med. 2013; 369(3):290-292.
40. Bloemberg G V, Keller P M, Stucki D, et al. Acquired Resistance to Bedaquiline and Delamanid in Therapy for Tuberculosis. N Engl J Med. 2015; 373(20):1986-1988.
41. Robasky K, Lewis N E, Church G M. The role of replicates for error mitigation in next-generation sequencing. Nat Rev Genet. 2014; 15(1):56-62.
42. Chen-Harris H, Borucki M K, Torres C, Slezak T R, Allen J E. Ultra-deep mutant spectrum profiling: improving sequencing accuracy using overlapping read pairs. BMC Genomics. 2013; 14:96.
43. Colman R E, Anderson J, Lemmer D, et al. Rapid Drug Susceptibility Testing of Drug-Resistant Mycobacterium tuberculosis Isolates Directly from Clinical Samples by Use of Amplicon Sequencing: a Proof-of-Concept Study. J Clin Microbiol. 2016; 54(8):2058-2067.
44. Metcalfe J Z, Streicher E, Theron G, et al. Cryptic Micro-heteroresistance Explains M. tuberculosis Phenotypic Resistance. Am J Respir Crit Care Med. 2017.
45. Gonzalo X, Claxton P, Brown T, Montgomery L, Fitzgibbon M, Laurenson I, Drobniewski F. True rifampicin resistance missed by the MGIT: prevalence of this pheno/genotype in the UK and Ireland after 18 month surveillance. Clin Microbiol Infect. 2017; 23(4):260-263.
46. Canetti G, Froman S, Grosset J, et al. Mycobacteria: Laboratory Methods for Testing Drug Sensitivity and Resistance. Bull World Health Organ. 1963; 29:565-578.
47. World Health Organization. 2018. Technical report on critical concentrations for TB drug susceptibility testing of medicines used in the treatment of drug-resistant TB.
48. Zhang Z, Lu J, Wang Y, Pang Y, Zhao Y. Automated liquid culture system misses isoniazid heteroresistance in Mycobacterium tuberculosis isolates with mutations in the promoter region of the inhA gene. Eur J Clin Microbiol Infect Dis. 2015; 34(3):555-560.
49. Folkvardsen D B, Thomsen V O, Rigouts L, et al. Rifampin heteroresistance in Mycobacterium tuberculosis cultures as detected by phenotypic and genotypic drug susceptibility test methods. J Clin Microbiol. 2013; 51(12):4220-4222.
50. Perez-Lago L, Comas I, Navarro Y, et al. Whole genome sequencing analysis of intrapatient microevolution in Mycobacterium tuberculosis: potential impact on the inference of tuberculosis transmission. J Infect Dis. 2014; 209(1):98-108.
51. Martin C J, Cadena A M, Leung V W, et al. Digitally Barcoding Mycobacterium tuberculosis Reveals In Vivo Infection Dynamics in the Macaque Model of Tuberculosis. MBio. 2017; 8(3).
52. Borrell S, Teo Y, Giardina F, et al. Epistasis between antibiotic resistance mutations drives the evolution of extensively drug-resistant tuberculosis. Evol Med Public Health. 2013; 2013(1):65-74.
53. Kaplan G, Post F A, Moreira A L, et al. Mycobacterium tuberculosis growth at the cavity surface: a microenvironment with failed immunity. Infect Immun. 2003; 71(12):7099-7108.
54. Moreno-Gamez S, Hill A L, Rosenbloom D I, Petrov D A, Nowak M A, Pennings P S. Imperfect drug penetration leads to spatial monotherapy and rapid evolution of multidrug resistance. Proc Natl Acad Sci USA. 2015; 112(22): E2874-2883.

We claim:

1. A method of detecting and/or quantifying a drug-resistant subpopulation of Mycobacterium tuberculosis in a sample, comprising:
obtaining an amplicon from the sample, wherein the amplicon comprises a region of interest in pncA (SEQ ID NO: 48), tlyA (SEQ ID NO: 49), gidB (SEQ ID NO: 50), rpsL (SEQ ID NO: 51), gyrB (SEQ ID NO: 52), embB (SEQ ID NO: 53), ahpC promoter, rplC (SEQ ID NO: 54), or a combination thereof, or a combination thereof, and the region of interest comprises a polymorphism associated with the drug-resistant subpopulation;
obtaining sequencing data by sequencing the amplicon on a Next Generation Sequencing (NGS) platform; and
detecting and/or quantifying a minor variant of the polymorphism, wherein the presence of the minor variant indicates the presence of the drug-resistant subpopulation.

2. The method of claim 1, wherein obtaining the amplicon uses a primer comprising a sequence at least 85% identical to an oligonucleotide selected from the group consisting of SEQ ID NOs: 1-43 or a complement thereof.

3. The method of claim 1, wherein the minor variant is selected from the group consisting of: a single nucleotide polymorphism (SNP), an insertion, a deletion, and combinations thereof.

4. The method of claim 1, wherein the region of interest comprises a polymorphism in pncA (SEQ ID NO: 48) associated with a pyrazinamide-resistant subpopulation, and the nucleotide is selected from the group consisting of SEQ ID NOs: 18-21.

5. The method of claim 4, wherein the minor variant comprises a deletion of 5′ GCACCC 3′, a deletion of 5′ GGGTGC 3′, or both.

6. The method of claim 1, wherein the region of interest comprises a polymorphism in tlyA (SEQ ID NO: 49) associated with a capreomycin-resistant subpopulation.

7. The method of claim 6, wherein the oligonucleotide is selected from the group consisting of SEQ ID NOs: 1-6 or a complement thereof.

8. The method of claim 1, wherein the region of interest comprises a polymorphism in gidB (SEQ ID NO: 50) associated with the streptomycin-resistant subpopulation.

9. The method of claim 8, wherein the oligonucleotide is selected from the group consisting of SEQ ID NOs: 7-10 or a complement thereof.

10. The method of claim 1, wherein the region of interest comprises a polymorphism in rpsL (SEQ ID NO: 51) associated with a streptomycin-resistant subpopulation.

11. The method of claim 10, wherein the oligonucleotide is selected from the group consisting of SEQ ID NOs: 11-14 or a complement thereof.

12. The method of claim 1, wherein the region of interest comprises a polymorphism in gyrB (SEQ ID NO: 52) associated with a quinolones-resistant subpopulation.

13. The method of claim 12, wherein the oligonucleotide is selected from the group consisting of SEQ ID NOs: 15-17 or a complement thereof.

14. The method of claim 1, wherein the region of interest comprises a polymorphism in pncA (SEQ ID NO: 48) associated with a pyrazinamide-resistant subpopulation, and the oligonucleotide is selected from the group consisting of SEQ ID NOs: 22-29 or a complement thereof.

15. The method of claim 1, wherein the region of interest comprises a polymorphism in embB (SEQ ID NO: 53) associated with an ethambutol-resistant subpopulation.

16. The method of claim 15, wherein the oligonucleotide is selected from the group consisting of SEQ ID NOs: 30-33 or a complement thereof.

17. The method of claim 1, further comprising administering to the subject a therapeutic agent based on the drug resistance of the Mycobacterium tuberculosis subpopulation in the sample.

18. The method of claim 17, wherein the therapeutic agent is selected from the group consisting of: an antibiotic, PA-824, OPC-67683, SQ109, TMC207, NAS-21, NAS-91, and combinations thereof.

19. A primer for detecting and/or quantifying a drug-resistant subpopulation of Mycobacterium tuberculosis in a sample, comprising
a sequence at least 85% identical to an oligonucleotide selected from the group consisting of SEQ ID NOs: 1-47 or a complement thereof; and
a label or a modified nucleotide.

20. A kit for detecting and/or quantifying a drug-resistant subpopulation of Mycobacterium tuberculosis in a sample, comprising:
a primer comprising a sequence at least 85% identical to an oligonucleotide selected from the group consisting of SEQ ID NOs: 1-47 or a complement thereof; and a label or a modified nucleotide; and
reagents for amplification of a genomic sample.
